(19) United States
(12) Patent Application Publication
(10) Pub. No.: US 2025/0307368 A1
(43) Pub. Date: Oct. 2, 2025

(54) AUTHENTICATION SUPPORT APPARATUS, AUTHENTICATION SUPPORT SYSTEM, AND AUTHENTICATION SUPPORT METHOD

(71) Applicants: NEC Corporation, Tokyo (JP); NATIONAL CENTER FOR GLOBAL HEALTH AND MEDICINE, Tokyo (JP)

(72) Inventors: Takafumi YOSHINAGA, Tokyo (JP); Yasushi OKADA, Tokyo (JP); Takumi TANAKA, Tokyo (JP); Shigeki KANO, Tokyo (JP); Shohei YAMAGUCHI, Tokyo (JP); Kengo MIYO, Tokyo (JP); Ryota NISHI, Tokyo (JP)

(73) Assignees: NEC Corporation, Tokyo (JP); NATIONAL CENTER FOR GLOBAL HEALTH AND MEDICINE, Tokyo (JP)

(21) Appl. No.: 19/086,290

(22) Filed: Mar. 21, 2025

(30) Foreign Application Priority Data
Mar. 28, 2024 (JP) 2024-052726

(51) Int. Cl. G06F 21/32; G16H 40/20; G16H 40/67

(52) U.S. Cl. CPC G06F 21/32; G16H 40/20; G16H 40/67

(57) ABSTRACT

An example object of the present disclosure is to reduce inconvenience of three-point authentication before administration of a medication to a patient. An authentication support apparatus includes: a first acquisition unit configured to acquire a wearer authentication result indicating whether or not a wearer is a predetermined medical practitioner based on an iris authentication result of the wearer who is wearing the eyewear-type wearable terminal; a second acquisition unit configured to acquire a patient authentication result based on authentication information of a patient imaged by the eyewear-type wearable terminal; and a third acquisition unit configured to acquire medication registration information indicating a correspondence between a medication specified based on medication information of the medication imaged by the eyewear-type wearable terminal and the patient who is an administration target.

### INCORPORATION BY REFERENCE

This application is based upon and claims the benefit of priority from Japanese patent application No. 2024-052726, filed on Mar. 28, 2024, the disclosure of which is incorporated herein in its entirety by reference.

### TECHNICAL FIELD

The present disclosure relates to an authentication support apparatus, an authentication support system, an authentication support method, and an authentication support program.

### BACKGROUND ART

In the case of a medical act of administering a medication to a patient, a person who intends to perform the medical act (a person scheduled to perform a medical act) performs so-called “three-point authentication” in the following (1) to (3) before the administration of the medication. Here, the “three-point authentication” is to perform (1) authentication that the person scheduled to perform the medical act is a qualified person for the medical act, (2) authentication of the patient as an individual, and (3) authentication that the patient authenticated in (2) is a target to be administered with the medication (a medication type, a dose, medication identification information, or the like). Then, the person scheduled to perform the medical act administers the medication to the patient after the three-point authentication is successful. In addition, the person scheduled to perform the medical act records a result of the three-point authentication.

Japanese Unexamined Patent Application Publication No. 2005-062994 discloses a technology related to a medical act determination system. The system according to Japanese Unexamined Patent Application Publication No. 2005-062994 performs face authentication by imaging a nurse with a camera, and comprehensively determines whether or not a correspondence among the nurse, a patient, and a medicine is correct by using a face authentication result, a patient-side determination apparatus, and a medication-side determination apparatus.

### SUMMARY

However, there is a problem that it is complicated for the person scheduled to perform the medical act to perform the three-point authentication before the administration of the medication to the patient. In particular, in the case of an emergency or the like, the inconvenience of the three-point authentication may increase a risk of a medical accident.

An example object of the present disclosure is to provide an authentication support apparatus, an authentication support system, an authentication support method, and an authentication support program for reducing inconvenience of three-point authentication before administration of a medication to a patient.

In a first example aspect, an authentication support apparatus includes:
a first acquisition unit configured to acquire a wearer authentication result indicating whether or not a wearer is a predetermined medical practitioner based on an iris authentication result of the wearer who is wearing the eyewear-type wearable terminal;
a second acquisition unit configured to acquire a patient authentication result based on authentication information of a patient imaged by the eyewear-type wearable terminal;
a third acquisition unit configured to acquire medication registration information indicating a correspondence between a medication specified based on medication information of the medication imaged by the eyewear-type wearable terminal and the patient who is an administration target; and
an output control unit configured to perform control to display, on a wearer screen of the eyewear-type wearable terminal, a three-point authentication result regarding permissibility of a medical act of administering the medication to the patient by the wearer based on the wearer authentication result, the patient authentication result, and the medication registration information.

In a second example aspect, an authentication support system includes:
an eyewear-type wearable terminal; and
an authentication support apparatus, in which
the eyewear-type wearable terminal is configured to transmit an iris image of a wearer who is wearing the eyewear-type wearable terminal, authentication information of an imaged patient, and medication information of an imaged medication to the authentication support apparatus,
the authentication support apparatus is configured to:
acquire a wearer authentication result indicating whether or not the wearer is a predetermined medical practitioner based on an iris authentication result for the iris image received from the eyewear-type wearable terminal;
acquire a patient authentication result based on the authentication information received from the eyewear-type wearable terminal;
acquire medication registration information indicating a correspondence between the medication specified based on the medication information received from the eyewear-type wearable terminal and the patient who is an administration target; and
transmit a three-point authentication result regarding permissibility of a medical act of administering the medication to the patient by the wearer to the eyewear-type wearable terminal based on the wearer authentication result, the patient authentication result, and the medication registration information, and
the eyewear-type wearable terminal is configured to display the three-point authentication result received from the authentication support apparatus on a wearer screen.

In a third example aspect, an authentication support method executed by a computer includes:
acquiring a wearer authentication result indicating whether or not a wearer is a predetermined medical practitioner based on an iris authentication result of the wearer who is wearing an eyewear-type wearable terminal;
acquiring a patient authentication result based on authentication information of a patient imaged by the eyewear-type wearable terminal;
acquiring medication registration information indicating a correspondence between a medication specified based on medication information of the medication imaged by the eyewear-type wearable terminal and the patient who is an administration target; and
performing control to display, on a wearer screen of the eyewear-type wearable terminal, a three-point authentication result regarding permissibility of a medical act of administering the medication to the patient by the wearer based on the wearer authentication result, the patient authentication result, and the medication registration information.

In a fourth example aspect, an authentication support program causes a computer to perform:
first acquisition processing of acquiring a wearer authentication result indicating whether or not a wearer is a predetermined medical practitioner based on an iris authentication result of the wearer who is wearing the eyewear-type wearable terminal;
second acquisition processing of acquiring a patient authentication result based on authentication information of a patient imaged by the eyewear-type wearable terminal;
third acquisition processing of acquiring medication registration information indicating a correspondence between a medication specified based on medication information of the medication imaged by the eyewear-type wearable terminal and the patient who is an administration target; and
output control processing of performing control to display, on a wearer screen of the eyewear-type wearable terminal, a three-point authentication result regarding permissibility of a medical act of administering the medication to the patient by the wearer based on the wearer authentication result, the patient authentication result, and the medication registration information.

An example advantage according to the above-described embodiments is reduction of inconvenience of three-point authentication before administration of a medication to a patient.

### BRIEF DESCRIPTION OF DRAWINGS

The above and other aspects, features and advantages of the present disclosure will become more apparent from the following description of certain exemplary embodiments when taken in conjunction with the accompanying drawings, in which:

FIG. 1 is a block diagram illustrating a configuration of an authentication support apparatus according to the present disclosure;

FIG. 2 is a flowchart illustrating a flow of an authentication support method according to the present disclosure;

FIG. 3 is a block diagram illustrating an overall configuration of an authentication support system according to the present disclosure;

FIG. 4 is a diagram for describing a concept of three-point authentication and medication administration performed by a medical practitioner wearing augmented reality (AR) glasses according to the present disclosure;

FIG. 5 is a block diagram illustrating a configuration of the AR glasses according to the present disclosure;

FIG. 6 is a block diagram illustrating a hardware configuration of the AR glasses according to the present disclosure;

FIG. 7 is a block diagram illustrating a configuration of an authentication support server according to the present disclosure;

FIG. 8 is a block diagram illustrating a hardware configuration of the authentication support server according to the present disclosure;

FIG. 9 is a sequence chart illustrating a flow of authentication support processing when a medical worker wears the AR glasses (normal mode) according to the present disclosure;

FIG. 10 is a diagram illustrating a display example of a wearer screen of the AR glasses when wearing the AR glasses (normal mode) according to the present disclosure;

FIG. 11 is a sequence chart illustrating a flow of authentication support processing at the start of the three-point authentication according to the present disclosure;

FIG. 12 is a diagram illustrating a display example of the wearer screen of the AR glasses after state transition to a three-point authentication execution state according to the present disclosure;

FIG. 13 is a diagram illustrating a display example of the wearer screen of the AR glasses after reading a face image of a patient and medication information according to the present disclosure;

FIG. 14 is a diagram illustrating an operation example for transitioning to the three-point authentication execution state according to the present disclosure;

FIG. 15 is a diagram illustrating an operation example for transitioning to the three-point authentication execution state according to the present disclosure;

FIG. 16 is a diagram illustrating an operation example for transitioning to the three-point authentication execution state according to the present disclosure;

FIG. 17 is a diagram illustrating a display example of the wearer screen of the AR glasses in a case where the three-point authentication has succeeded according to the present disclosure;

FIG. 18 is a diagram illustrating a display example of the wearer screen of the AR glasses in a case where the three-point authentication has failed according to the present disclosure;

FIG. 19 is a diagram for describing a concept of the three-point authentication performed by the medical practitioner wearing the AR glasses according to the present disclosure; and

FIG. 20 is a block diagram illustrating a configuration of the AR glasses according to the present disclosure.

### EXAMPLE EMBODIMENTS

Hereinafter, embodiments of the present disclosure will be described in detail with reference to the drawings. In the drawings, the same or corresponding elements are denoted by the same reference numerals, and repeated description is omitted as necessary for clarity of description.

### First Embodiment

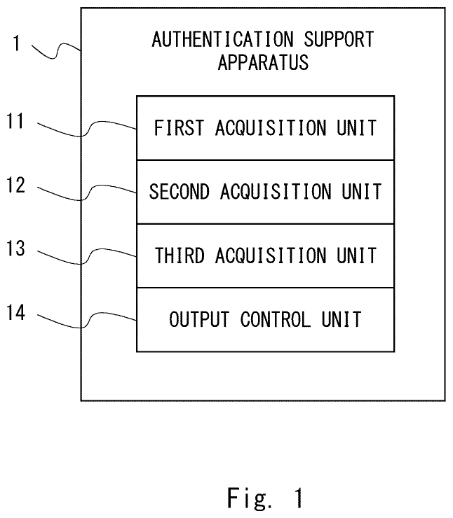

FIG. 1 is a block diagram illustrating a configuration of an authentication support apparatus 1. The authentication support apparatus 1 is an information processing apparatus for supporting a predetermined medical practitioner to perform three-point authentication regarding permissibility of a medical act of administering a medication registered in advance to a patient. Here, the “medical practitioner” is a person qualified to administer (for example, inject) a medication to a patient among medical workers. The medical practitioner is, for example, a person who has a qualification such as a doctor or a nurse. It is assumed that the medical practitioner wears an eyewear-type wearable terminal capable of performing iris authentication for a wearer at the time of the three-point authentication and the medication administration. In addition, the authentication support apparatus 1 may be any one of the eyewear-type wearable terminal itself or a computer server communicably connected to the eyewear-type wearable terminal.

The authentication support apparatus 1 includes a first acquisition unit 11, a second acquisition unit 12, a third acquisition unit 13, and an output control unit 14. The first acquisition unit 11 acquires a wearer authentication result indicating whether or not the wearer is a predetermined medical practitioner based on an iris authentication result of the wearer who is wearing the eyewear-type wearable terminal. The second acquisition unit 12 acquires a patient authentication result based on authentication information of the patient imaged by the eyewear-type wearable terminal. The third acquisition unit 13 acquires medication registration information specified based on medication information of the medication imaged by the eyewear-type wearable terminal. The medication registration information indicates a correspondence between the medication and the patient who is an administration target.

The output control unit 14 performs control to display a three-point authentication result on a wearer screen of the eyewear-type wearable terminal based on the wearer authentication result, the patient authentication result, and the medication registration information. The three-point authentication result is a result of the authentication regarding the permissibility of the medical act of administering the medication to the patient by the wearer.

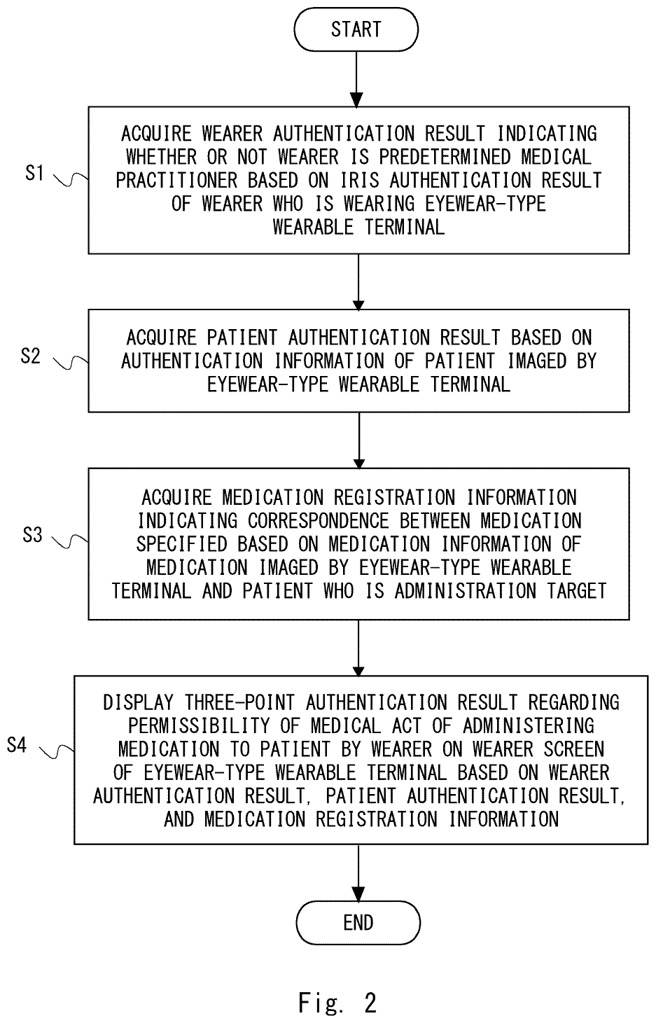

FIG. 2 is a flowchart illustrating a flow of an authentication support method. First, the first acquisition unit 11 acquires the wearer authentication result indicating whether or not the wearer is the predetermined medical practitioner based on the iris authentication result of the wearer who is wearing the eyewear-type wearable terminal (S1). Next, the second acquisition unit 12 acquires the patient authentication result based on the authentication information of the patient imaged by the eyewear-type wearable terminal (S2). Then, the third acquisition unit 13 acquires the medication registration information indicating the correspondence between the medication specified based on the medication information of the medication imaged by the eyewear-type wearable terminal and the patient who is the administration target (S3). Note that the order of steps S1 to S3 is not limited thereto. For example, two or more of steps S1 to S3 may be performed in parallel.

After steps S1 to S3, the output control unit 14 displays the three-point authentication result regarding the permissibility of the medical act of administering the medication to the patient by the wearer on the wearer screen of the eyewear-type wearable terminal based on the wearer authentication result, the patient authentication result, and the medication registration information (S4).

As described above, in the present embodiment, (1) the wearer is subjected to personal authentication (identity confirmation) by the iris authentication performed on the wearer (a person scheduled to perform the medical act) wearing the eyewear-type wearable terminal, and it is possible to perform authentication that the wearer is a qualified person for a predetermined medical act from attribute information or the like of the wearer specified according to the personal authentication. In a case where the iris authentication information of the qualified person for the predetermined medical act is registered in a database of the iris authentication, success in the iris authentication of the wearer can be regarded as authentication that the wearer is the qualified person for the predetermined medical act.

In addition, in the present embodiment, personal authentication of the patient can be performed by (2) patient authentication performed based on the authentication information specified from the image of the patient imaged by the wearer using the eyewear-type wearable terminal. Then, in the present embodiment, (3) the medication registration information is specified based on the medication information of the medication imaged by the wearer using the eyewear-type wearable terminal, and thus, the specified medication registration information can be acquired. In the three-point authentication, it is determined that the authentication has succeeded when the wearer authentication result of (1) indicates that the person scheduled to perform the medical act is the qualified person, and the patient authenticated in (2) and the medication imaged in (3) are associated with the medication registration information acquired in (3). In the present embodiment, the three-point authentication is performed based on the above (1) to (3), so that the three-point authentication result is displayed on the wearer screen of the eyewear-type wearable terminal. Therefore, the wearer can confirm whether or not the wearer can perform the medical act of administering the medication to the patient in front of the wearer. The three-point authentication result can be registered in an external medical information system. As described above, with the technology according to the present disclosure, it is possible to reduce inconvenience of the three-point authentication before administration of a medication to a patient.

The authentication support apparatus 1 includes a processor, a memory, and a storage device as components (not illustrated). In addition, the storage device stores, for example, a computer program in which processing of the authentication support method of FIG. 2 is implemented. Then, the processor reads the computer program or the like from the storage device into the memory and executes the computer program. As a result, the processor implements functions of the first acquisition unit 11, the second acquisition unit 12, the third acquisition unit 13, and the output control unit 14.

Alternatively, each component of the authentication support apparatus 1 may be implemented by dedicated hardware. In addition, some or all of the components of each apparatus may be implemented by general-purpose or dedicated circuitry, a processor, or a combination thereof. These components may be implemented by a single chip or may be implemented by a plurality of chips connected via a bus. Some or all of the components of each apparatus may be implemented by a combination of the above-described circuitry or the like and a program. Furthermore, as the processor, a central processing unit (CPU), a graphics processing unit (GPU), a field-programmable gate array (FPGA), a quantum processor (quantum computer control chip), or the like can be used.

Furthermore, in a case where some or all of the components of the authentication support apparatus 1 are implemented by a plurality of information processing apparatuses, circuitries, and the like, the plurality of information processing apparatuses, circuitries, and the like may be arranged in a centralized manner or in a distributed manner. For example, the information processing apparatuses, the circuitries, or the like may be implemented in the form of a client server system, a cloud computing system, or the like in which the information processing apparatuses, the circuitries, or the like are connected to each other via a communication network. Furthermore, the function of the authentication support apparatus 1 may be provided in a software as a service (SaaS) format.

### Second Embodiment

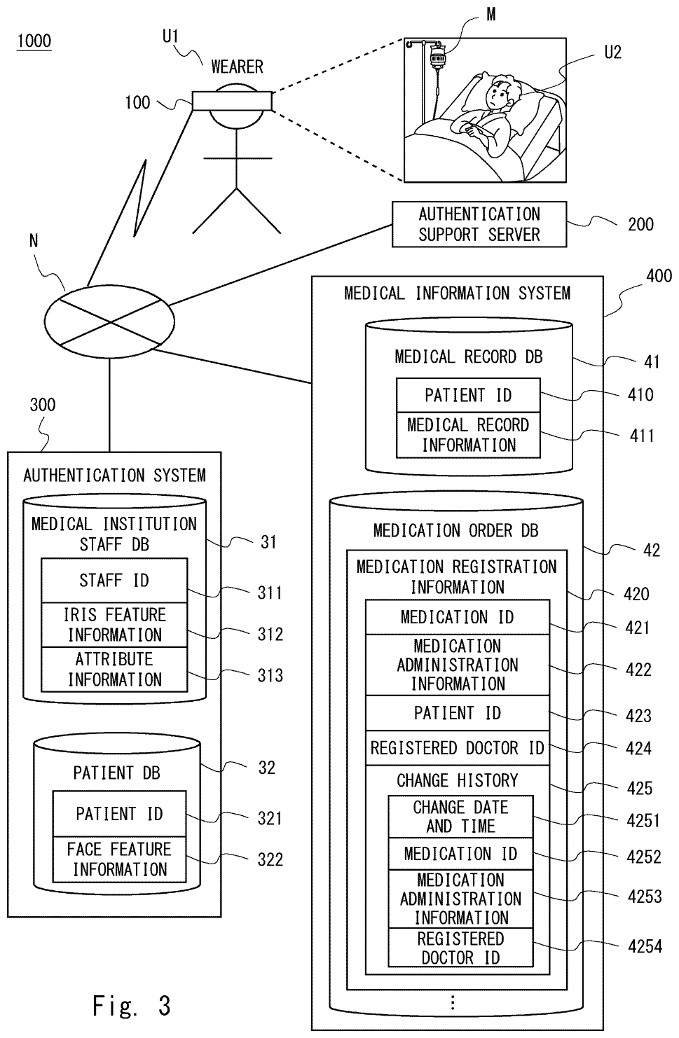

FIG. 3 is a block diagram illustrating an overall configuration of an authentication support system 1000. The authentication support system 1000 is an information system for supporting three-point authentication performed before a wearer U1 wearing augmented reality (AR) glasses 100 administers a predetermined medication M to a patient U2. The authentication support system 1000 includes the AR glasses 100, an authentication support server 200, an authentication system 300, and a medical information system 400. However, the authentication support system 1000 may include at least the AR glasses 100 and the authentication support server 200. The AR glasses 100, the authentication support server 200, the authentication system 300, and the medical information system 400 are communicably connected via a network N. Here, the network N is a wired or wireless communication line.

The wearer U1 is a person scheduled to perform a medical act, the person wearing the AR glasses 100 and being present in the vicinity of the patient U2 and the medication M. The patient U2 is an inpatient on a hospital bed or an outpatient. Then, the patient U2 is a person to whom a predetermined medication is to be administered by a predetermined medical practitioner. The medication M is a medication to be administered to the patient U2. The medication M is, for example, an intravenous drip, and an information code capable of specifying medication information is provided thereon.

The AR glasses 100 are an example of an eyewear-type wearable terminal that is worn by the person scheduled to perform the medical act for the three-point authentication and includes a screen which also serves as a lens and through which light from an external peripheral space is transmitted. In addition, the AR glasses 100 display various types of display information described below on a field-of-view region of a real space visually recognized by the wearer through light transmitted from the outside, in a superimposed manner. The AR glasses 100 may be, for example, a head mounted display (HMD). A detailed configuration of the AR glasses 100 is described below.

The authentication support server 200 is an information processing apparatus for supporting the three-point authentication by using the authentication system 300, the medical information system 400, and the like according to information received from the AR glasses 100. A detailed configuration of the authentication support server 200 is described below.

The authentication system 300 is an information system that performs authentication processing for identity confirmation of a medical institution staff (in particular, the person scheduled to perform the medical act) and a patient. The authentication system 300 performs iris authentication as the identity confirmation of the person scheduled to perform the medical act, and performs face authentication, which is one of biometric authentication, as an example of the identity confirmation of the patient. The authentication system 300 may perform biometric authentication other than the face authentication or identity confirmation processing other than the biometric authentication as the identity confirmation of the patient. The authentication system 300 includes at least a medical institution staff database (DB) 31 and a patient DB 32.

The medical institution staff DB 31 is a database for managing staff information of a predetermined medical institution. For example, the medical institution staff DB 31 manages a staff ID 311, iris feature information 312, and attribute information 313 in association with each other. The staff ID 311 is identification information of a staff of the predetermined medical institution. The iris feature information 312 is a feature vector including a set of feature points of the iris of the eye of the staff. For example, the iris feature information 312 is a set of pieces of information calculated as a feature amount for a pattern such as a shape indicating the feature of the iris in an iris region detected from an image including the eyes of the corresponding staff. The attribute information 313 is information indicating an attribute including qualification information (for example, a doctor or a nurse) of the corresponding staff.

The patient DB 32 is a database that manages a patient ID 321 and face feature information 322 in association with each other. The patient DB 32 may manage attribute information (a name or the like) of the patient other than the face feature information 322 in association with the patient ID 321. The patient ID 321 is identification information of the patient U2. The face feature information 322 is a feature vector including a set of feature points of the face of the patient corresponding to the patient ID 321. For example, the face feature information 322 is a set of pieces of information calculated using, as a feature amount, a distance or the like between a plurality of feature points indicating face features (the eyes, the nose, the mouth, and the like) in a face region detected from a face image of the corresponding patient.

The authentication system 300 determines whether the iris authentication has succeeded or failed according to a matching degree between iris feature information (query) based on an iris image included in an iris authentication request received via the network N and a plurality of pieces of (master) iris feature information 312 in the medical institution staff DB 31. In a case where the iris authentication has succeeded, the authentication system 300 specifies the staff ID 311 and the attribute information 313 associated with the iris feature information 312 for which the iris authentication has succeeded, includes the specified staff ID 311 and the specified attribute information 313 in the iris authentication result, and transmits the iris authentication result as a response to a request source.

In addition, the authentication system 300 determines whether the face authentication has succeeded or failed based on a matching degree between face feature information (query) based on a face image included in a face authentication request received via the network N and a plurality of pieces of (master) face feature information 322 in the patient DB 32. In a case where the face authentication has succeeded, the authentication system 300 specifies the patient ID 321 (and the attribute information) associated with the face feature information 322 for which the face authentication has succeeded, includes the specified patient ID 321 (and the specified attribute information) in a face authentication result, and transmits the face authentication result as a response to a request source.

The medical information system 400 is an information system for managing medical information such as a medical record and medication order information. The medical information system 400 includes at least a medical record DB 41 and a medication order DB 42. The medical record DB 41 is a database that manages medical record information of a patient. The medical record DB 41 manages a patient ID 410 and medical record information 411 in association with each other. The patient ID 410 may be associated with two or more pieces of medical record information 411. The patient ID 410 is information equivalent to the patient ID 321. Therefore, the patient ID 321 of the specific patient U2 is the same as the patient ID 410 or information that can uniquely specify the patient ID 410.

The medication order DB 42 is a database that manages one or more pieces of medication registration information 420. The medication registration information 420 is information regarding a medication to be administered to a specific patient by a doctor, the information being registered in advance. The medication registration information 420 is, for example, information in which a medication ID 421, medication administration information 422, a patient ID 423, a registered doctor ID 424, and a change history 425 are associated with each other. The medication ID 421 is identification information of the medication. The medication administration information 422 is information regarding a scheduled administration date and time and a dosage of the corresponding medication. The patient ID 423 is identification information of a patient who is an administration target with the corresponding medication. The patient ID 423 is information equivalent to the patient ID 410 described above. The registered doctor ID 424 is identification information of the doctor who has registered the medication administration information 422 in order to administer the corresponding medication to the corresponding patient. The change history 425 is a change history of the medication ID 421 and the medication administration information 422 that have been registered for the patient corresponding to the patient ID 423. The change history 425 is, for example, information in which a change date and time 4251, a medication ID 4252, medication administration information 4253, and a registered doctor ID 4254 are associated with each other. The change date and time 4251 is information indicating a date and time (date, hour, minute, second, and the like) when the medication registration information 420 has been changed. The medication ID 4252 is the medication ID 421 before the change. The medication administration information 4253 is the medication administration information 422 before the change. The registered doctor ID 4254 is the registered doctor ID 424. Therefore, in a case where the medication registration information 420 has been changed, the registered doctor ID 424 is identification information of the doctor who has changed the medication registration information 420. In addition, the change history 425 may include two or more change histories including at least the latest change history.

The medical information system 400 searches the medical record DB 41 by using a patient ID included in a medical record information acquisition request received via the network N as a search key, and transmits the searched medical record information 411 as a response to a request source. In addition, the medical information system 400 searches the medication order DB 42 by using a medication ID included in a medication registration information acquisition request received via the network N as a search key, and transmits the searched medication registration information 420 as a response to the request source.

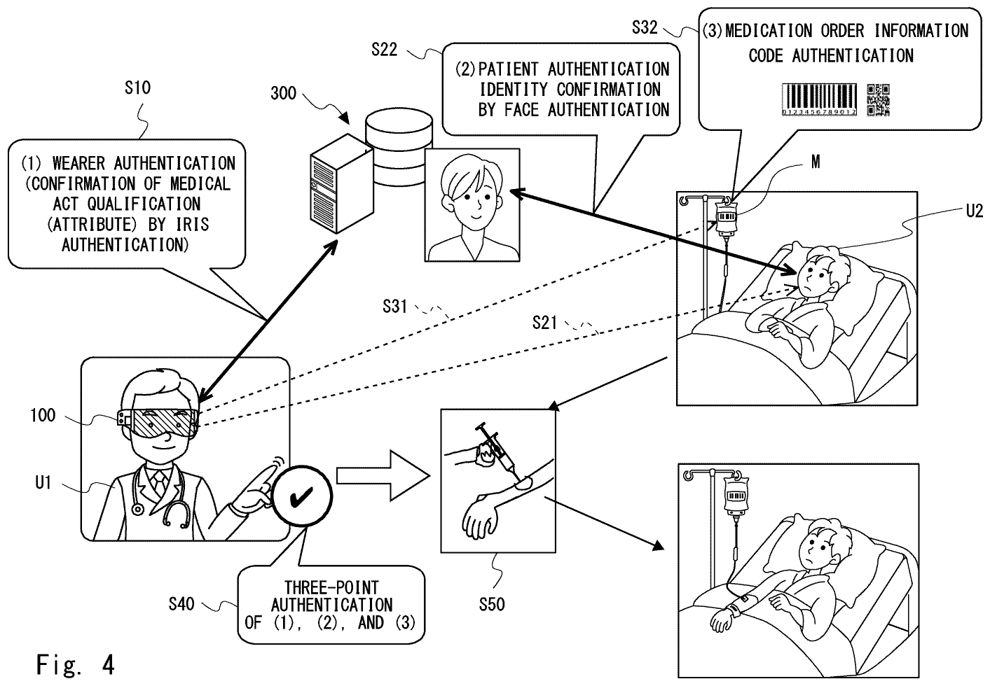

FIG. 4 is a diagram for describing a concept of the three-point authentication and the medication administration performed by the medical practitioner (wearer U1) wearing the AR glasses 100. First, the AR glasses 100 perform “(1) wearer authentication” after the wearer U1 wears the AR glasses 100 (S10). That is, the AR glasses 100 confirm medical act qualification (attribute) by the iris authentication with the authentication system 300 (via the authentication support server 200). In a case where the wearer authentication has succeeded, the wearer U1 directs the face (gaze) of the wearer U1 to the vicinity of the face of the patient U2. Therefore, an external camera (not illustrated) of the AR glasses 100 captures a face image of the patient U2 (S21). Then, the authentication support server 200 performs the identity confirmation (“(2) patient authentication”) by the face authentication based on the face image of the patient U2 using the authentication system 300 (S22). In addition, the wearer U1 directs the face (gaze) of the wearer U1 to the vicinity of the information code attached to the medication M. Therefore, the external camera of the AR glasses 100 images the information code of the medication M (S31). The information code is, for example, a bar code which is a one-dimensional code, a quick response (QR) code (registered trademark) which is a two-dimensional code, or the like, but is not limited thereto. Then, the authentication support server 200 specifies the medication order information (medication registration information) based on the medication ID analyzed from the information code imaged and read using the authentication system 300, and performs “(3) code authentication” (S32). After steps S10, S22, and S32, the AR glasses 100 display a result (here, “OK”) of the three-point authentication performed in (1), (2), and (3) on a wearer screen (not illustrated) (S40). As a result, the wearer U1 confirms that the three-point authentication has succeeded, and injects the medication M to the patient U2 (S50).

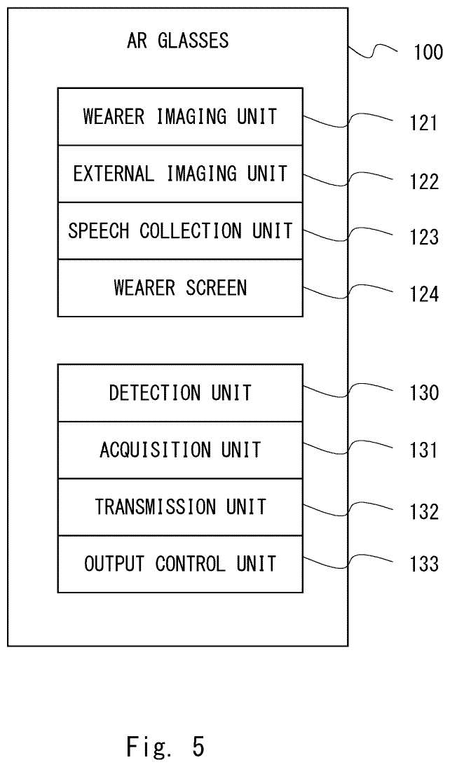

FIG. 5 is a block diagram illustrating a configuration of the AR glasses 100. The AR glasses 100 include a wearer imaging unit 121, an external imaging unit 122, a speech collection unit 123, a wearer screen 124, a detection unit 130, an acquisition unit 131, a transmission unit 132, and an output control unit 133.

The wearer imaging unit 121 is a camera that images a region including the iris of the eye of the wearer U1, a circuitry or software that controls the camera, or the like. The wearer imaging unit 121 outputs an iris image including the iris of the wearer U1 to the acquisition unit 131.

The external imaging unit 122 is a camera that images an imaging region including a display region (field-of-view region) of the wearer screen 124, a circuitry or software that controls the camera, or the like. The external imaging unit 122 images at least an area in front of the wearer U1 and outputs the captured image to the acquisition unit 131. For example, the external imaging unit 122 captures the face image of the patient U2, the information code of the medication, an information code for state transition, and the like. The external imaging unit 122 may record the captured image in an internal storage unit (not illustrated).

The speech collection unit 123 is a microphone that collects a speech around the AR glasses 100, a circuitry or software that controls the microphone, or the like. The speech collection unit 123 collects a speech uttered by the wearer U1 and a speech uttered by the patient U2. Therefore, the speech collection unit 123 collects a speech of a conversation between the wearer U1, who is a medical worker, and the patient U2 present in the vicinity of the AR glasses 100 during a medical interview. The speech collection unit 123 may record collected speech information, at least speech information, in the storage unit.

The wearer screen 124 is a display that covers at least a part of the field-of-view region of the wearer U1 of the AR glasses 100. In addition, the wearer screen 124 displays a display image (video) output by the output control unit 133 in a superimposed manner in the field-of-view region through which light from an external peripheral space is transmitted.

The detection unit 130 detects a predetermined operation of the wearer U1 through the AR glasses 100. Examples of the detected predetermined operation include the following. The detection unit 130 may detect a predetermined voice command by performing speech recognition for the speech collected by the speech collection unit 123. Alternatively, the detection unit 130 may detect a region indicated by the gaze of the wearer U1 on the wearer screen 124 by the wearer imaging unit 121 or a gaze sensor (not illustrated). For example, in a case where predetermined display information (an icon image or the like) is displayed on the wearer screen 124, the detection unit 130 detects that the gaze of the wearer U1 is directed to a region of the display information. Alternatively, the detection unit 130 may detect a motion (a gesture or the like) of the hand or finger of the wearer U1 for the wearer screen 124 by the wearer imaging unit 121 or the external imaging unit 122. For example, in a case where the external imaging unit 122 images the hand or finger of the wearer U1, the AR glasses 100 display an image (for example, a polygon image) corresponding to the hand or finger of the wearer U1 on the wearer screen 124. In a case where the predetermined display information (the icon image or the like) is displayed on the wearer screen 124, the detection unit 130 may detect an operation of pressing the display information by the finger of the wearer U1. Alternatively, the detection unit 130 may detect the gesture of the hand or finger of the wearer U1 imaged by the external imaging unit 122. Alternatively, the detection unit 130 may detect the information codes imaged by the external imaging unit 122. Alternatively, the detection unit 130 may detect the above-described predetermined voice command, the motion (the gesture or the like) of the hand or finger of the wearer U1, and a combination or order of the imaged information codes.

The acquisition unit 131 is an example of the first acquisition unit 11, the second acquisition unit 12, and the third acquisition unit 13 described above. The acquisition unit 131 acquires the iris image of the wearer U1 from the wearer imaging unit 121, the face image and the information code of the medication or the like of the patient U2 from the external imaging unit 122, and speech data of the wearer U1 or the like from the speech collection unit 123.

The transmission unit 132 may transmit the iris authentication request including the iris image of the wearer U1 acquired by the acquisition unit 131 to the authentication support server 200 via the network N. Then, the acquisition unit 131 acquires the wearer authentication result based on the iris authentication result of the authentication system 300 in the authentication support server 200 from the authentication support server 200.

In addition, the transmission unit 132 may transmit the face authentication request including the face image of the patient U2 acquired by the acquisition unit 131 to the authentication support server 200 via the network N. In addition, the transmission unit 132 may transmit the information code of the medication acquired by the acquisition unit 131 to the authentication support server 200 via the network N. Then, the acquisition unit 131 acquires the three-point authentication result performed in the authentication support server 200 from the authentication support server 200.

In addition, the transmission unit 132 transmits a detection notification for the predetermined operation detected by the detection unit 130 to the authentication support server 200 via the network N. Then, the acquisition unit 131 acquires an instruction for state transition to a three-point authentication execution state from the authentication support server 200. The transmission unit 132 may transmit information itself regarding the predetermined operation detected by the detection unit 130 (speech data, an operation content, or the like) to the authentication support server 200.

The output control unit 133 may display the past medical record information of the patient U2 acquired by the acquisition unit 131 on the wearer screen 124. In addition, the output control unit 133 may display instruction information for transition to the three-point authentication execution state on the wearer screen 124. In addition, the output control unit 133 displays a patient check field and a medication check field on the wearer screen 124 according to the transition to the three-point authentication execution state. For example, in a case where an instruction for transition to the three-point authentication execution state is received from the authentication support server 200, the output control unit 133 may display the patient check field and the medication check field on the wearer screen 124. Once the acquisition unit 131 acquires the face image of the patient U2, the output control unit 133 displays the fact that the patient has been read in the patient check field on the wearer screen 124. Once the acquisition unit 131 acquires the information code of the medication, the output control unit 133 displays the fact that the medication has been read in the medication check field on the wearer screen 124. Once the acquisition unit 131 acquires the three-point authentication result, the output control unit 133 displays the three-point authentication result on the wearer screen 124.

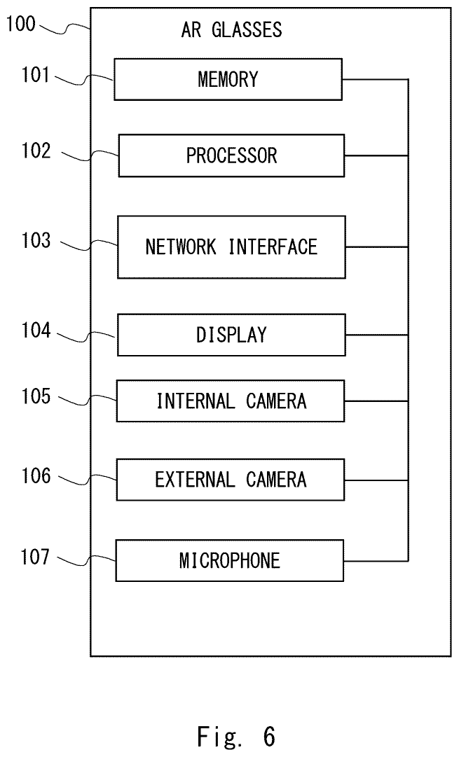

FIG. 6 is a block diagram illustrating a hardware configuration of the AR glasses 100. The AR glasses 100 include a memory 101, a processor 102, a network interface 103, a display 104, an internal camera 105, an external camera 106, and a microphone 107.

The memory 101 is implemented by a combination of a volatile memory and a nonvolatile memory. The volatile memory is, for example, a volatile storage device such as a random access memory (RAM), and is a storage region for temporarily holding information during operation of the processor 102. The nonvolatile memory is, for example, a nonvolatile storage device such as a flash memory. The memory 101 stores at least a computer program in which a part of processing of a medical record support method of the AR glasses 100 according to the present disclosure is implemented.

The processor 102 is a control apparatus that controls each component of the AR glasses 100. The processor 102 reads and executes software (computer program) from the memory 101. Therefore, the processor 102 implements the functions of the detection unit 130, the acquisition unit 131, the transmission unit 132, and the output control unit 133. That is, the processor 102 performs a part of the processing of the authentication support method according to the present disclosure. The processor 102 may be, for example, a microprocessor, a multi processing unit (MPU), or a CPU. Further, the processor 102 may include a plurality of processors.

The network interface 103 may be used to communicate with network nodes. The network interface 103 may include, for example, a network interface card (NIC) conforming to IEEE 802.3 series. IEEE represents Institute of Electrical and Electronics Engineers. Furthermore, the network interface 103 may include a wireless local area network (LAN), a wired LAN, Wi-Fi (registered trademark), Bluetooth (registered trademark), and the like.

The display 104 is a display apparatus that displays information instructed from the processor 102. The display 104 is, for example, a screen such as a liquid crystal display or an organic electro-luminescence (EL) display. In particular, the display 104 transmits light from the outside and corresponds to the above-described wearer screen 124.

In response to an instruction from the processor 102, the internal camera 105 images a region including the iris of the eye of the wearer of the AR glasses 100, and outputs the captured iris image to the processor 102. The internal camera 105 corresponds to the above-described wearer imaging unit 121.

In response to an instruction from the processor 102, the external camera 106 images an external peripheral space including an area in front of the AR glasses 100, and outputs the captured image to the processor 102. The external camera 106 is, for example, a charge coupled device (CCD) image sensor, a complementary metal oxide semiconductor (CMOS) sensor, or the like. The external camera 106 includes one or more imaging devices. The external camera 106 corresponds to the above-described external imaging unit 122.

The microphone 107 is a sound collector that collects sound around the AR glasses 100.

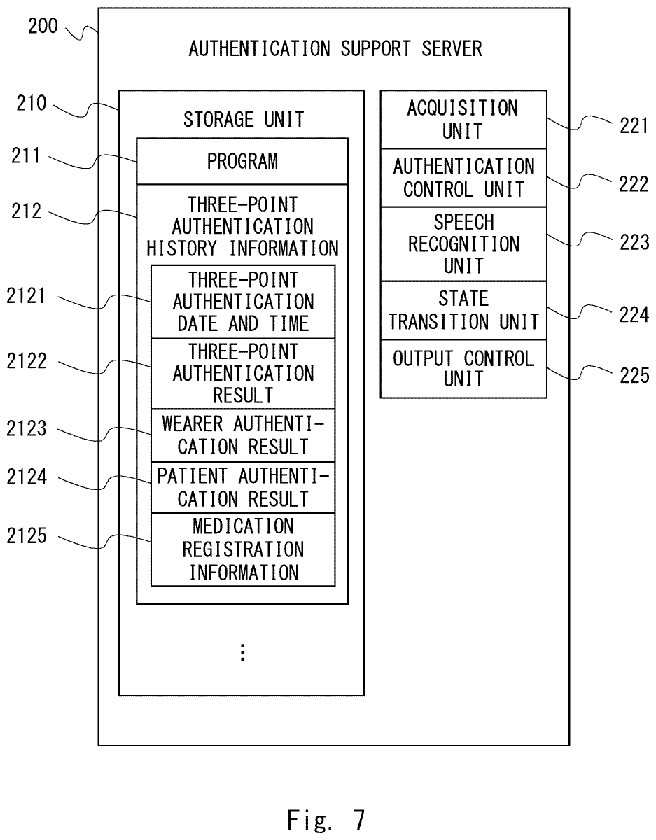

FIG. 7 is a block diagram illustrating a configuration of the authentication support server 200. The authentication support server 200 is an information processing apparatus for supporting the three-point authentication performed before the wearer U1 of the AR glasses 100 administers the predetermined medication M to the patient U2 by using the authentication system 300, the medical information system 400, and the like according to information received from the AR glasses 100. The authentication support server 200 is an example of the authentication support apparatus 1 described above. The authentication support server 200 includes a storage unit 210, an acquisition unit 221, an authentication control unit 222, a speech recognition unit 223, a state transition unit 224, and an output control unit 225.

The storage unit 210 includes, for example, a nonvolatile storage device such as a hard disk or a flash memory, and a memory such as a RAM, that is, a volatile storage device. The storage unit 210 stores a program 211 and three-point authentication history information 212. The program 211 is a computer program in which at least a part of authentication support processing according to the present disclosure is implemented. The three-point authentication history information 212 is history information of the three-point authentication result. The three-point authentication history information 212 is, for example, information in which a three-point authentication date and time 2121, a three-point authentication result 2122, a wearer authentication result 2123, a patient authentication result 2124, and medication registration information 2125 are associated with each other. The three-point authentication date and time 2121 is information indicating a date and time when the three-point authentication according to the present disclosure is performed (for example, year, month, day, hour, minute, and second). The three-point authentication result 2122 is information indicating whether the entire three-point authentication has been succeeded or failed.

The wearer authentication result 2123 is an authentication result indicating that the wearer U1 of the AR glasses 100 is a qualified person for the medical act. The wearer authentication result 2123 may include at least information including whether or not the attribute information 313 of the wearer (staff ID 311) for which the iris authentication has succeeded indicates a qualified person for the medical act of administering the medication M, that is, information indicating whether the wearer authentication has succeeded or failed. For example, in a case where the iris authentication has succeeded, the wearer authentication result 2123 may include the staff ID 311 and the attribute information 313, and may include a result of determining whether or not the attribute information 313 indicates a qualified person for the medical act. In a case where only qualified persons for the medical act are registered in the medical institution staff DB 31, the wearer authentication result 2123 may include at least information indicating whether the iris authentication has succeeded or failed, and in a case where the iris authentication has succeeded, the wearer authentication result 2123 may include the staff ID 311.

The patient authentication result 2124 is the face authentication result of the patient imaged by the AR glasses 100. The patient authentication result 2124 includes at least information indicating whether the face authentication has succeeded or failed, and in a case where the face authentication has succeeded, the patient authentication result 2124 may include the patient ID 321.

The medication registration information 2125 is the medication registration information 420 specified based on the medication information of the medication imaged by the AR glasses 100. That is, a case where the three-point authentication result 2122 indicates success can be said to be a case where three points are satisfied: the wearer authentication result 2123 indicates that the wearer authentication has succeeded, the patient authentication result 2124 indicates the patient authentication has succeeded, and the patient ID 423 included in the medication registration information 2125 matches the patient ID included in the patient authentication result 2124.

The acquisition unit 221 is an example of the first acquisition unit 11, the second acquisition unit 12, and the third acquisition unit 13 described above. The acquisition unit 221 acquires the wearer authentication result indicating whether or not the wearer U1 is the predetermined medical practitioner based on the iris authentication result for the iris image of the wearer U1 received from the AR glasses 100. For example, the acquisition unit 221 may acquire, as the wearer authentication result, a determination result obtained by determining whether the wearer U1 is the predetermined medical practitioner based on the iris authentication result of the authentication control unit 222 described below.

In addition, the acquisition unit 221 acquires the patient authentication result based on the authentication information received from the AR glasses 100. For example, the acquisition unit 221 may acquire, as the patient authentication result, the face authentication result of the authentication control unit 222 described below. Specifically, the acquisition unit 221 may acquire the patient authentication result by controlling the patient authentication based on the authentication information detected from the image related to the patient U2 captured by the AR glasses 100.

In addition, the acquisition unit 221 acquires the medication registration information indicating a correspondence between the medication M specified based on the medication information received from the AR glasses 100 and the patient U2 who is the administration target. Specifically, the acquisition unit 221 may acquire the medication registration information by specifying the medication registration information corresponding to the medication information detected from the image related to the medication M captured by the AR glasses 100.

The authentication control unit 222 controls the iris authentication for the iris image of the wearer U1 captured by the wearer imaging unit 121 of the AR glasses 100 by using the authentication system 300. Then, the authentication control unit 222 determines whether or not the wearer U1 is the predetermined medical practitioner based on the iris authentication result. In addition, the authentication control unit 222 controls the face authentication for the face image of the patient U2 captured by the external imaging unit 122 of the AR glasses 100 by using the authentication system 300. In addition, the authentication control unit 222 analyzes the information code of the medication received from the AR glasses 100 to specify the medication ID of the medication information. Then, the authentication control unit 222 specifies and acquires the medication registration information 420 in which the specified medication ID is included in the medication ID 421 or the medication ID 4252 from the medication order DB 42 of the medical information system 400.

In addition, the authentication control unit 222 performs the three-point authentication based on the acquired wearer authentication result, patient authentication result, and medication registration information. Specifically, the authentication control unit 222 determines that the three-point authentication has succeeded in a case where the wearer authentication result 2123 indicates that the wearer authentication has succeeded, the patient authentication result 2124 indicates that the patient authentication has succeeded, and it is confirmed that the patient ID 423 included in the medication registration information 2125 matches the patient ID included in the patient authentication result 2124.

The speech recognition unit 223 converts the speech data into text information by performing the speech recognition on the speech data collected by the AR glasses 100. In particular, the speech recognition unit 223 may perform the speech recognition by distinguishing speakers based on at least one of a volume and a speech collection direction in the speech collection unit 123 of the AR glasses 100.

In a case where the predetermined operation of the wearer U1 through the AR glasses 100 is detected, the state transition unit 224 transitions an operation state of the AR glasses 100 to the three-point authentication execution state in which at least the authentication information and the medication information can be acquired. Further, the state transition unit 224 performs state transition to the three-point authentication execution state in a case where the wearer authentication result indicates that the wearer U1 is the predetermined medical practitioner, and the predetermined operation of the wearer U1 has been detected. In a case where utterance of a predetermined speech by the wearer U1 is detected as the predetermined operation, the state transition unit 224 performs transition to the three-point authentication execution state.

In a case where the pressing operation by the wearer U1 for the instruction information for transition to the three-point authentication execution state displayed on the wearer screen 124 is detected as the predetermined operation, the state transition unit 224 performs transition to the three-point authentication execution state. For example, in a case where the utterance of the predetermined speech by the wearer U1 through the AR glasses 100 is detected as the predetermined operation, the state transition unit 224 may perform transition to the three-point authentication execution state. Alternatively, in a case where the pressing operation by the wearer U1 through the AR glasses 100 for the instruction information for transition to the three-point authentication execution state displayed on the wearer screen 124 is detected as the predetermined operation, the state transition unit 224 may perform transition to the three-point authentication execution state. Alternatively, in a case where the utterance of the predetermined speech by the wearer U1 after the wearer U1 directs the gaze through the AR glasses 100 for the instruction information for transition to the three-point authentication execution state displayed on the wearer screen 124 is detected as the predetermined operation, the state transition unit 224 may perform transition to the three-point authentication execution state. Alternatively, in a case where a change in gaze of the wearer U1 toward the information code for the three-point authentication posted at an administration place for the medication M through the AR glasses 100 is detected as the predetermined operation, the state transition unit 224 may perform transition to the three-point authentication execution state.

The output control unit 225 is an example of the output control unit 14 described above. In a case where the image related to the patient U2 or the medication M is captured by the AR glasses 100 after the transition to the three-point authentication execution state, the output control unit 225 may perform control to display, on the wearer screen 124, the fact that image is captured.

In addition, the output control unit 225 transmits, to the AR glasses 100, the three-point authentication result 2122 regarding the permissibility of the medical act of administering the medication M to the patient U2 by the wearer U1 based on the wearer authentication result 2123, the patient authentication result 2124, and the medication registration information 2125. That is, the output control unit 225 performs control to display the three-point authentication result 2122 on the wearer screen 124 of the AR glasses 100.

Specifically, in a case where the three-point authentication result 2122 indicates that the medical act of administering the medication M to the patient U2 by the wearer U1 of the AR glasses 100 is permitted, the output control unit 225 transmits the fact to the AR glasses 100 together with the three-point authentication history information 212. Furthermore, in a case where the three-point authentication result 2122 indicates the medical act is not permitted, the output control unit 225 performs control to display, together with the three-point authentication result 2122, display information describing the reason why the medical act is not permitted on the wearer screen 124 of the AR glasses 100. Furthermore, in a case where the three-point authentication result 2122 indicates that the medical act is not permitted, the output control unit 225 may operate the AR glasses 100 such that the wearer U1 can perceive the fact that the medical act is not permitted.

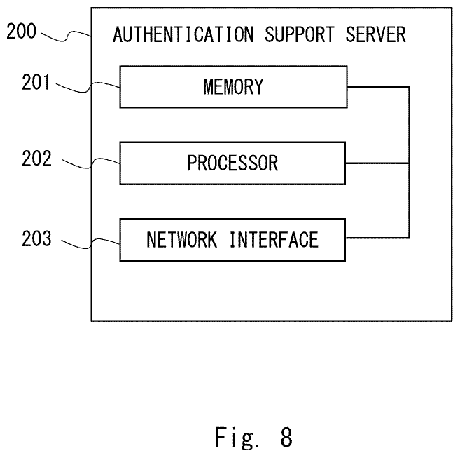

FIG. 8 is a block diagram illustrating a hardware configuration of the authentication support server 200. The authentication support server 200 includes a memory 501, a processor 202, and a network interface 203.

The memory 201 stores at least a computer program (for example, the program 211) in which at least a part of the processing of the authentication support method of the authentication support server 200 according to the present disclosure is implemented. Further, the memory 201 may store the three-point authentication history information 212. Other configurations of the memory 201 are similar to those of the memory 101. The processor 202 is a control apparatus that controls each component of the authentication support server 200. The processor 202 reads and executes software (computer program) from the memory 201. As a result, the processor 202 implements the functions of the acquisition unit 221, the authentication control unit 222, the speech recognition unit 223, the state transition unit 224, and the output control unit 225. That is, the processor 202 performs at least a part of the authentication support method in the authentication support server 200 according to the present disclosure. Other configurations of the processor 202 are similar to those of the processor 102. The network interface 203 has a configuration similar to that of the network interface 103.

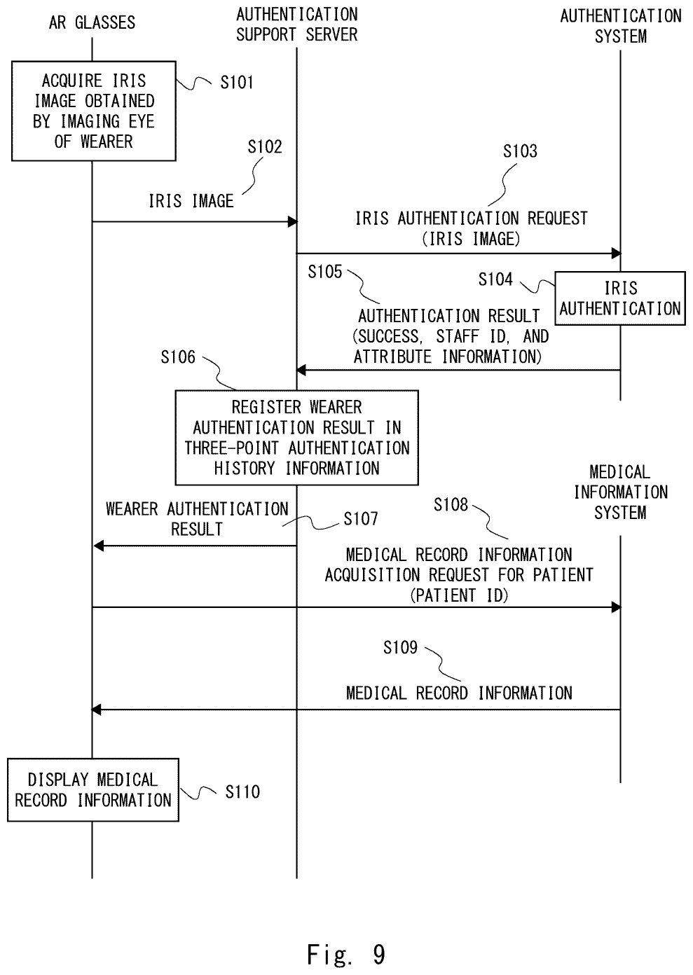

FIG. 9 is a sequence chart illustrating a flow of the authentication support processing when the medical worker wears the AR glasses 100 (normal mode). First, the person scheduled to perform the medical act, who intends to administer the medication M to the patient U2, wears the AR glasses 100 and becomes the wearer U1 in order to perform the three-point authentication before the administration of the medication M. At this time, the AR glasses 100 acquire the iris image obtained by imaging the eye of the wearer U1 by the wearer imaging unit 121 (S101). Then, the AR glasses 100 transmit the iris authentication request including the acquired iris image of the wearer U1 to the authentication support server 200 via the network N (S102). Then, the authentication support server 200 transmits the received iris authentication request to the authentication system 300 via the network N (S103).

The authentication system 300 performs the iris authentication of the patient U2 based on the received iris authentication request (S104).

Specifically, the authentication system 300 detects the iris region from the iris image included in the iris authentication request, and calculates the feature amount for the pattern such as the shape indicating the feature of the iris from the detected iris region. Then, the authentication system 300 extracts a feature vector including a set of a plurality of calculated feature amounts as the (query) iris feature information. The iris authentication request may include the iris feature information extracted by the AR glasses 100 or the authentication support server 200 instead of the iris image.

Then, the authentication system 300 compares the query iris feature information with the plurality of pieces of (master) iris feature information 312 in the medical institution staff DB 31, and calculates a matching score indicating the matching degree as the degree of coincidence. For example, the authentication system 300 may calculate the matching score to be higher as a distance between the feature vectors of the master and the query is shorter. Then, the authentication system 300 determines that the iris authentication has succeeded in a case where the degree of coincidence is equal to or higher than a threshold, and determines that the iris authentication has failed in a case where the degree of coincidence is lower than the threshold. The authentication system 300 includes, in the iris authentication result, a determination result of the iris authentication. In addition, in a case where the iris authentication has succeeded, the authentication system 300 specifies the staff ID 311 and the attribute information 313 associated with the iris feature information 312 for which the iris authentication has succeeded. Then, the authentication system 300 includes, in the iris authentication result, the specified staff ID 311 and the specified attribute information 313. Thereafter, the authentication system 300 transmits the iris authentication result as a response to the authentication support server 200 via the network N (S105). Hereinafter, for convenience of explanation, it is assumed that the wearer U1 has succeeded in the iris authentication. Therefore, the iris authentication result includes information indicating that the iris authentication has succeeded, the staff ID 311, and the attribute information 313.

Then, the authentication support server 200 determines whether or not the attribute information 313 included in the received iris authentication result is a qualification for the medical act of administering the medication M. That is, the authentication support server 200 determines that the wearer U1 who has succeeded in the iris authentication is a qualified person for the medical act, and sets the determination result as the wearer authentication result. Hereinafter, for convenience of explanation, it is assumed that the wearer U1 is determined to be a qualified person for the medical act.

Therefore, the authentication support server 200 generates the wearer authentication result 2123 including the staff ID 311 of the wearer U1 who has succeeded in the iris authentication, and registers the three-point authentication history information 212 including the wearer authentication result 2123 in the storage unit 210 (S106). Then, the authentication support server 200 transmits the wearer authentication result 2123 to the AR glasses 100 via the network N (S107). In response to the reception of the wearer authentication result 2123, the AR glasses 100 operate in a normal mode and make various functions available to the wearer U1.

Then, in response to the reception of the wearer authentication result 2123, the AR glasses 100 transmit the medical record information acquisition request for the patient U2 to the medical information system 400 via the network N (S108). At this time, the acquisition request may include, for example, the patient ID of the patient U2. For example, it is assumed that the wearer U1 grasps in advance the name and the patient ID of the patient U2 who is the administration target of the medication M.

Then, the medical information system 400 searches the medical record DB 41 by using the patient ID included in the received acquisition request as the search key, and transmits the searched medical record information 411 as a response to the AR glasses 100 (S109). The AR glasses 100 may transmit and receive the medical record information acquisition request and the medical record information to and from the medical information system 400 via the authentication support server 200.

Then, the AR glasses 100 receive the medical record information of the patient U2 from the medical information system 400 via the network N, and display the received latest medical record information on the wearer screen 124 (S110).

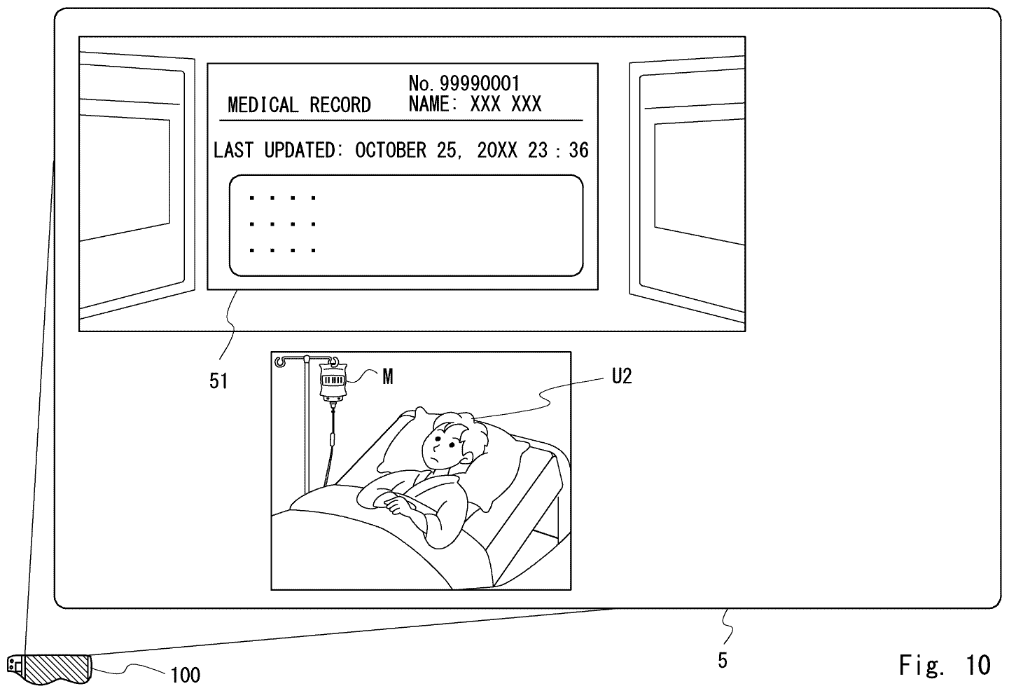

FIG. 10 is a diagram illustrating a display example of a wearer screen 5 when wearing the AR glasses 100 (normal mode). For example, in the wearer screen 5, the patient U2 and the medication M are transmitted through external light of the AR glasses 100 on a lower side of the screen, and medical record information 51 and the like are displayed on an upper side of the screen. The display example in the normal mode is not limited thereto.

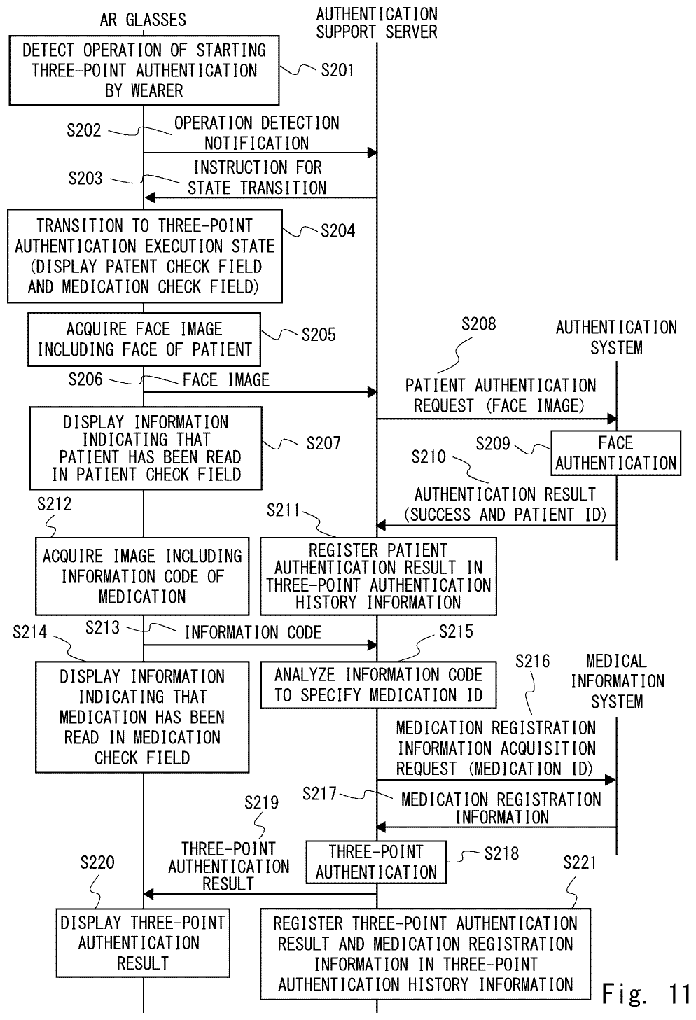

FIG. 11 is a sequence chart illustrating a flow of the authentication support processing at the start of the three-point authentication. In the normal mode of the AR glasses 100, the wearer U1 performs an operation of starting the three-point authentication. Then, the AR glasses 100 detect the operation of starting the three-point authentication by the wearer U1 (S201). A plurality of patterns that can be taken as the operation of starting the three-point authentication are described below. The AR glasses 100 transmit an operation detection notification to the authentication support server 200 via the network N (S202). In response to the received operation detection notification, the authentication support server 200 transmits the instruction for state transition to the three-point authentication execution state to the AR glasses 100 via the network N (S203). Then, the AR glasses 100 transition to the three-point authentication execution state (S204). For example, the AR glasses 100 display the patient check field and the medication check field on the wearer screen 124. Furthermore, in a case where the transition to the three-point authentication execution state has been performed, the AR glasses 100 may be capable of acquiring the face image by the face recognition or may be capable of acquiring the information code by code recognition from the image captured by the external imaging unit 122. In a case where the operation of starting the three-point authentication has been detected, the AR glasses 100 may perform state transition to the three-point authentication execution state by internal processing without transmitting and receiving the operation detection notification and the instruction for state transition to and from the authentication support server 200.

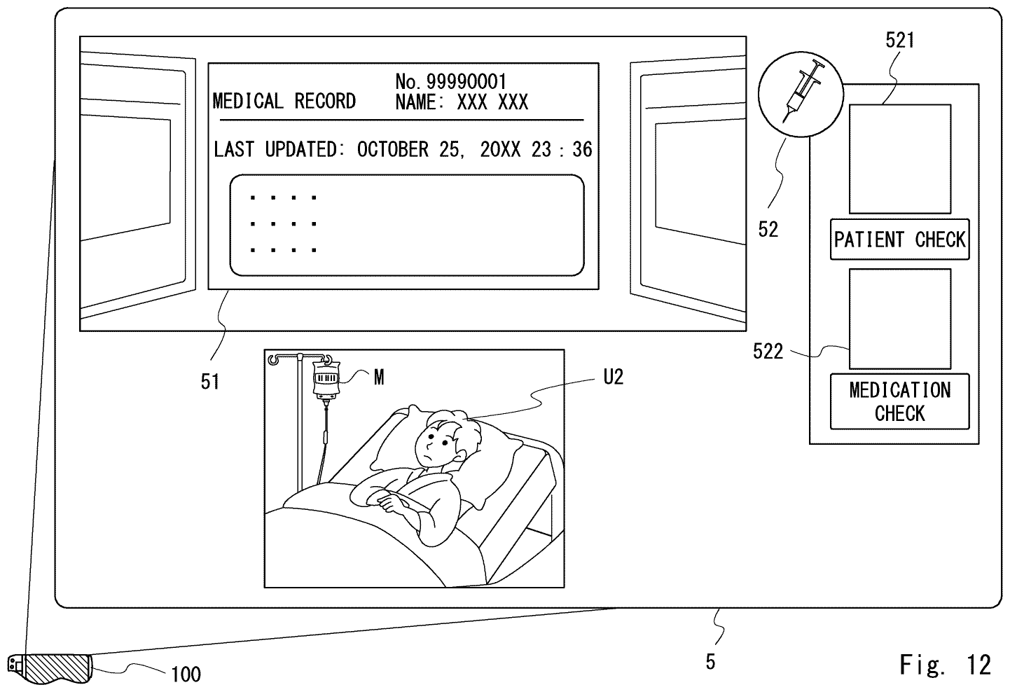

FIG. 12 is a diagram illustrating a display example of the wearer screen 5 of the AR glasses 100 after the state transition to the three-point authentication execution state. FIG. 12 illustrates an example in which the wearer screen 5 displays, for example, a three-point authentication mode image 52, a patient check field 521, and a medication check field 522 on the right side. The three-point authentication mode image 52 is an example of a display image indicating that an operation mode of the AR glasses 100 is the three-point authentication execution state. The patient check field 521 is a display field for displaying the fact that the face image of the patient for the patient authentication has been captured in a case where the face image has been captured. The medication check field 522 is a display field for displaying the fact that the information code of the medication has been imaged in a case where the information code has been imaged. At this time, since the state transition to the three-point authentication execution state has just been made, the patient check field 521 and the medication check field 522 are blank. The display example in the three-point authentication execution state is not limited thereto.

After step S204 in FIG. 11, the wearer U1 directs the face of the wearer U1 toward the face of the patient U2. Therefore, the AR glasses 100 image the face of the patient U2. That is, the AR glasses 100 image a region including the face of the patient U2 by the external imaging unit 122, and acquire the face image including the face of the patient U2 (S205). Then, the AR glasses 100 transmit the face authentication request including the face image of the patient U2 to the authentication support server 200 via the network N (S206). At this time, the AR glasses 100 may display information indicating that the patient has been read in the patient check field 521 (S207).

In response to step S206, the authentication support server 200 transmits the received face authentication request to the authentication system 300 via the network N (S208). Then, the authentication system 300 performs the face authentication of the patient U2 based on the received face authentication request (S209). Specifically, the authentication system 300 detects the face region from the face image included in the face authentication request, extracts a plurality of feature points indicating the face features (the eyes, the nose, the mouth, and the like) of the person from the detected face region, and calculates a distance between the respective feature points as the feature amount. Then, the authentication system 300 extracts the feature vector including a set of a plurality of calculated feature amounts as the (query) face feature information. The face authentication request may include the face feature information extracted by the AR glasses 100 or the authentication support server 200 instead of the face image.

Then, the authentication system 300 compares the query face feature information with the plurality of pieces of (master) face feature information 322 in the patient DB 32, and calculates a matching score indicating the matching degree as the degree of coincidence. For example, the authentication system 300 may calculate the matching score to be higher as a distance between the feature vectors of the master and the query is shorter. Then, the authentication system 300 determines that the face authentication has succeeded in a case where the degree of coincidence is equal to or higher than a threshold, and determines that the face authentication has failed in a case where the degree of coincidence is lower than the threshold. The authentication system 300 includes, in the face authentication result, a determination result of the face authentication. In addition, in a case where the face authentication has succeeded, the authentication system 300 specifies the patient ID 321 associated with the face feature information 322 for which the face authentication has succeeded. Then, the authentication system 300 includes, in the face authentication result, the specified patient ID 321. Thereafter, the authentication system 300 transmits the face authentication result as a response to the authentication support server 200 via the network N (S210). Hereinafter, for convenience of explanation, it is assumed that the patient U2 has succeeded in the face authentication. Therefore, the face authentication result includes information indicating that the face authentication has succeeded and the patient ID.

Then, the authentication support server 200 receives the face authentication result from the authentication system 300 via the network N. The authentication support server 200 confirms that the patient U2 has succeeded in the face authentication from the received face authentication result, and acquires the patient ID included in the face authentication result. Then, the authentication support server 200 generates the patient authentication result 2124 including the acquired patient ID, and registers the three-point authentication history information 212 including the patient authentication result 2124 in the storage unit 210 (S211).

Further, the wearer U1 directs the face of the wearer U1 toward the medication M. Therefore, the AR glasses 100 image the information code attached (posted) to the medication M. That is, the AR glasses 100 image a region including the information code of the medication M by the external imaging unit 122, and acquire an image including the information code of the medication M (S212). Then, the AR glasses 100 transmit a code authentication request for the medication order information including the information code to the authentication support server 200 via the network N (S213). At this time, the AR glasses 100 may display information indicating that the medication has been read in the medication check field 522 (S214).

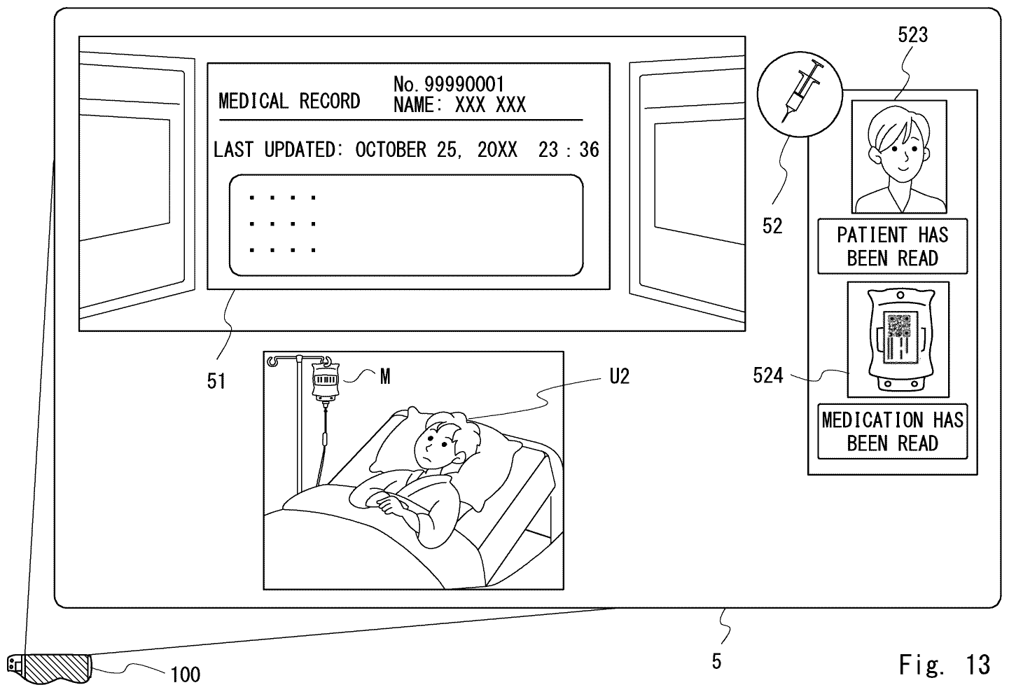

FIG. 13 is a diagram illustrating a display example of the wearer screen 5 of the AR glasses 100 after reading the face image of the patient U2 and the medication information of the medication M. FIG. 13 illustrates an example in which the wearer screen 5 displays, for example, a patient check field 523 updated from the patient check field 521 in FIG. 12 described above and a medication check field 524 updated from the medication check field 522 on the right side. The patient check field 523 is a display example indicating that the patient has been read. The medication check field 524 is a display example indicating that the medication has been read. However, the display examples are not limited thereto.

In response to step S213 in FIG. 11, the authentication support server 200 analyzes the information code included in the received code authentication request to specify the medication ID (S215). Then, the authentication support server 200 transmits the medication registration information acquisition request including the specified medication ID to the medical information system 400 via the network N (S216). Then, the medical information system 400 searches the medication order DB 42 by using the medication ID included in the received acquisition request as the search key. At this time, the medical information system 400 determines whether or not the medication ID of the search key is included in any one of the medication ID 421 and the medication ID 4252 in the medication registration information 420 in the medication order DB 42. Then, the medical information system 400 transmits the searched medication registration information 420 as a response to the authentication support server 200 (S217).

Thereafter, the authentication support server 200 performs the three-point authentication (S218). Specifically, the authentication support server 200 determines whether or not the patient ID 423 included in the received medication registration information 420 matches the patient ID included in the patient authentication result 2124. Further, the authentication support server 200 determines whether or not the medication ID specified in step S215 matches the medication ID 421 or the medication ID 4252 included in the medication registration information 420 received in step S217. In a case where the specified medication ID matches the latest medication ID 421 and the patient IDs match in the above determination, the authentication support server 200 determines that the medication authentication (medication code authentication) has succeeded. On the other hand, in a case where the specified medication ID does not match the latest medication ID 421, that is, in a case where the specified medication ID matches the past medication ID 4252 in the change history, the authentication support server 200 determines that the medication authentication has failed. Therefore, in a case where the wearer authentication result 2123 indicates that the wearer authentication has succeeded and the medication authentication (medication code authentication) has succeeded, the authentication support server 200 determines that the three-point authentication has succeeded, and generates the three-point authentication result including the fact. On the other hand, in other cases, the authentication support server 200 determines that the three-point authentication has failed, and generates the three-point authentication result including the fact. Specifically, the authentication support server 200 additionally registers the three-point authentication date and time 2121, the three-point authentication result 2122, and the medication registration information 2125 in the three-point authentication history information 212 (S221).

After step S218, the authentication support server 200 transmits the three-point authentication result 2122 to the AR glasses 100 via the network N (S219). Then, the AR glasses 100 display the received three-point authentication result 2122 on the wearer screen 124 (S220). An order of steps of processing in steps S205 to S211 and steps S212 to S217 may be changed, or the steps of processing may be performed in parallel.

Here, examples of the operation of starting the three-point authentication according to the present disclosure will be described in Examples 2-1 to 2-4 below. However, the operation of starting the three-point authentication is not limited thereto.

### Example 2-1: Voice Command

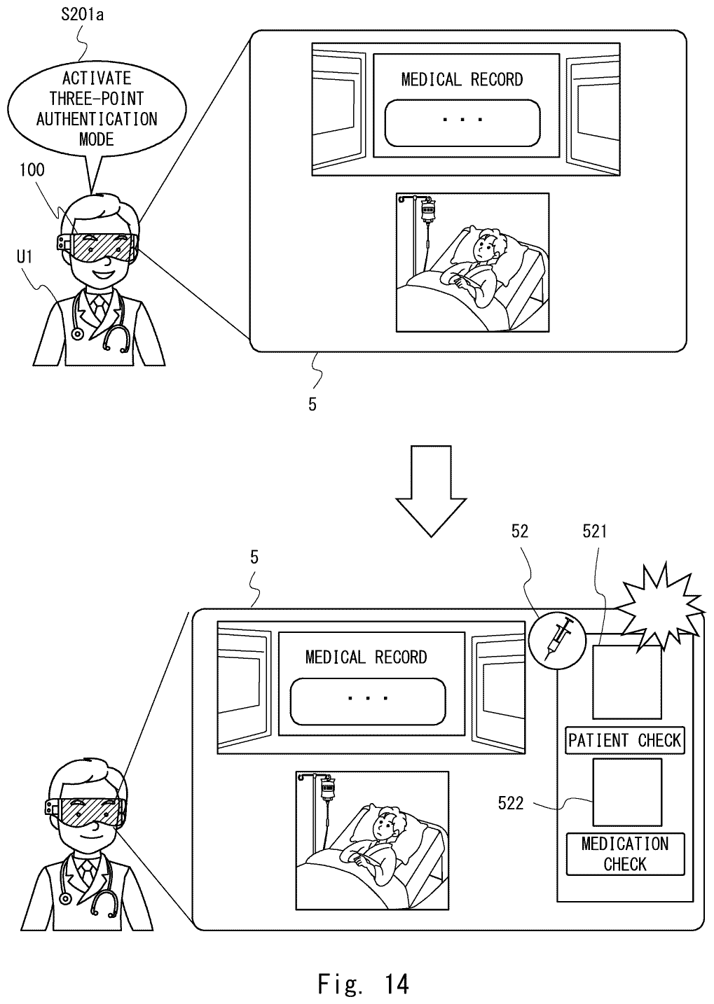

For example, the utterance of the predetermined speech by the wearer U1 can be used as the operation of starting the three-point authentication. FIG. 14 is a diagram illustrating an operation example for transitioning to the three-point authentication execution state. The wearer U1 wearing the AR glasses 100 utters “activate three-point authentication mode” in the normal mode (S201a). The AR glasses 100 may detect the speech data collected by the speech collection unit 123 as the operation of starting the three-point authentication by the wearer U1 in a case where the speech data is recognized as “activate three-point authentication mode”. An example of a phrase of the utterance (the voice command for transitioning to the three-point authentication execution state) is not limited thereto. Then, as described above, the AR glasses 100 transition to the three-point authentication execution state, and displays the three-point authentication mode image 52, the patient check field 521, and the medication check field 522 on the wearer screen 5.

### Example 2-2: Pressing of Mode Switching Icon

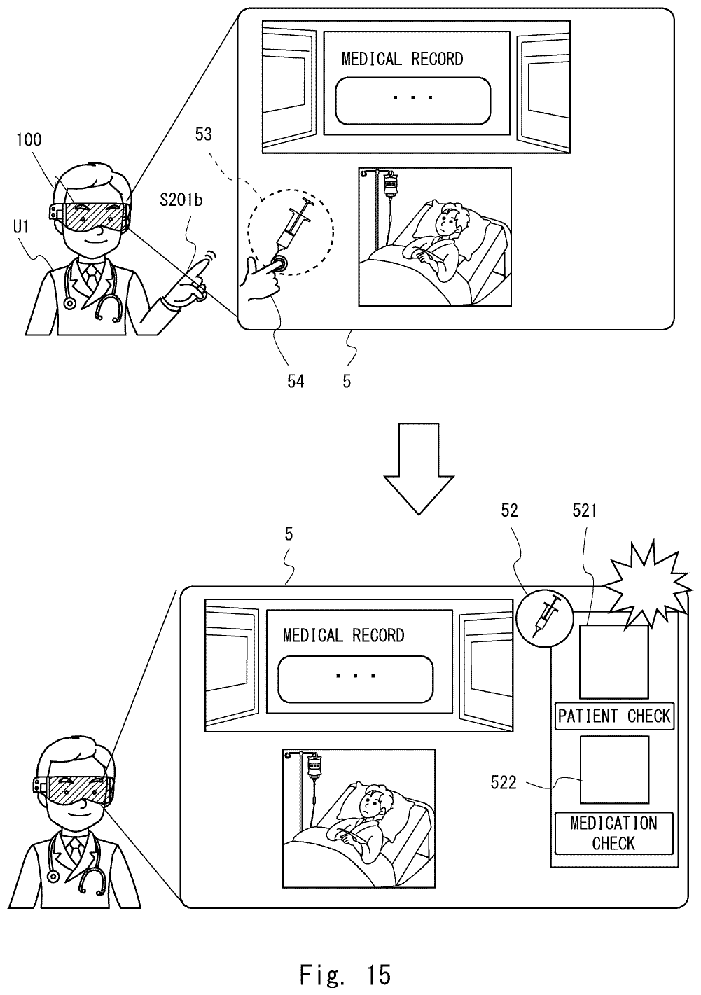

In addition, the pressing operation by the wearer U1 for the instruction information for transition to the three-point authentication execution state displayed on the wearer screen 5 can be detected as the operation of starting the three-point authentication. FIG. 15 is a diagram illustrating an operation example for transitioning to the three-point authentication execution state. First, the AR glasses 100 display a mode switching icon 53 on the wearer screen 5 in a menu or the like in the normal mode. The mode switching icon 53 is an example of the instruction information for transition to the three-point authentication execution state. In addition, the AR glasses 100 may display the mode switching icon 53 on the wearer screen 5 by an operation of the wearer U1 or the like. In this state (normal mode), the wearer U1 wearing the AR glasses 100 moves the finger of the wearer U1 to press the mode switching icon 53 on the wearer screen 5 (S201b). Specifically, when the hand including the finger of the wearer U1 is reflected in an imaging range of the external imaging unit 122, the AR glasses 100 display a finger motion image 54 of the wearer U1 on the wearer screen 5. Then, the wearer U1 moves the hand and the finger of the wearer U1 so as to move the finger motion image 54 to a position overlapping the mode switching icon 53. Then, for example, the wearer U1 moves the hand and the finger such that the finger motion image 54 of the wearer U1 presses the mode switching icon 53. Then, the AR glasses 100 detect the pressing operation by the wearer U1 on the mode switching icon 53 by analyzing the captured image of the external imaging unit 122. In this case, the AR glasses 100 may detect the pressing operation as the operation of starting the three-point authentication by the wearer U1. Then, as described above, the AR glasses 100 transition to the three-point authentication execution state, and displays the three-point authentication mode image 52, the patient check field 521, and the medication check field 522 on the wearer screen 5. The pressing operation for the mode switching icon is not limited thereto. Furthermore, the operation of starting the three-point authentication may be the gesture of the wearer U1. For example, the AR glasses 100 may detect a motion in which the wearer U1 puts two fingers together as the operation of starting the three-point authentication.

### Example 2-3: Gaze Toward Mode Switching Icon+Voice Command

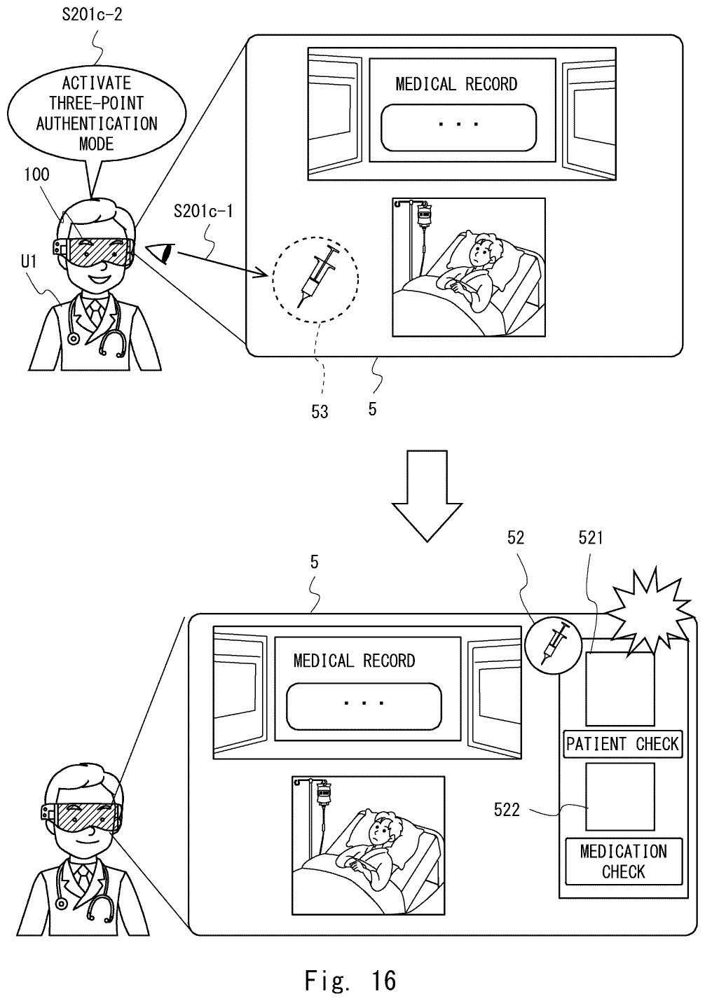

In addition, the utterance of the predetermined speech by the wearer U1 after the wearer U1 directs the gaze through the AR glasses 100 for the instruction information for transition to the three-point authentication execution state displayed on the wearer screen 5 can be detected as the operation of starting the three-point authentication. FIG. 16 is a diagram illustrating an operation example for transitioning to the three-point authentication execution state. First, the AR glasses 100 display the above-described mode switching icon 53 on the wearer screen 5 in the menu or the like in the normal mode. In this state (normal mode), the wearer U1 wearing the AR glasses 100 directs the gaze of the wearer U1 to the mode switching icon 53 (on the wearer screen 5) for a predetermined time (S201c-1). The predetermined time may be, for example, within several seconds. At this time, the AR glasses 100 detect that the gaze of the wearer U1 is directed to the mode switching icon 53 on the wearer screen 5 for the predetermined time by the wearer imaging unit 121. This state may be expressed as a state in which the wearer U1 locks the mode switching icon 53. In this state, the wearer U1 utters “activate three-point authentication mode” (S201c-2). In a case where step S201c-2 is performed after step S201c-1, the AR glasses 100 may detect the utterance as the operation of starting the three-point authentication by the wearer U1. Then, as described above, the AR glasses 100 transition to the three-point authentication execution state, and displays the three-point authentication mode image 52, the patient check field 521, and the medication check field 522 on the wearer screen 5. In a case where the gaze of the wearer U1 locks the mode switching icon 53 (that is, only step S201c-1), the AR glasses 100 may detect the gaze as the operation of starting the three-point authentication by the wearer U1. However, by performing step S201c-2 after step S201c-1, it is possible to prevent erroneous transition to the three-point authentication execution state when the wearer U1 unintentionally directs the gaze to the mode switching icon 53. In addition, it is possible to reduce time and effort of the wearer U1 to perform an operation (for example, cancellation operation) of returning from the three-point authentication execution state to the normal mode after erroneous transition to the three-point authentication execution state.

### Example 2-4: Imaging of Information Code for Three-Point Authentication Posted at Administration Place for Medication

In addition, a change in gaze of the wearer U1 toward the information code for the three-point authentication posted at the administration place for the medication M through the AR glasses 100 can be detected as the operation of starting the three-point authentication. In this case, the AR glasses 100 may analyze the information code imaged by the external imaging unit 122 and detect the change in gaze of the wearer U1 toward the information code for the three-point authentication as the operation of starting the three-point authentication. Then, as described above, the AR glasses 100 transition to the three-point authentication execution state, and displays the three-point authentication mode image 52, the patient check field 521, and the medication check field 522 on the wearer screen 5.

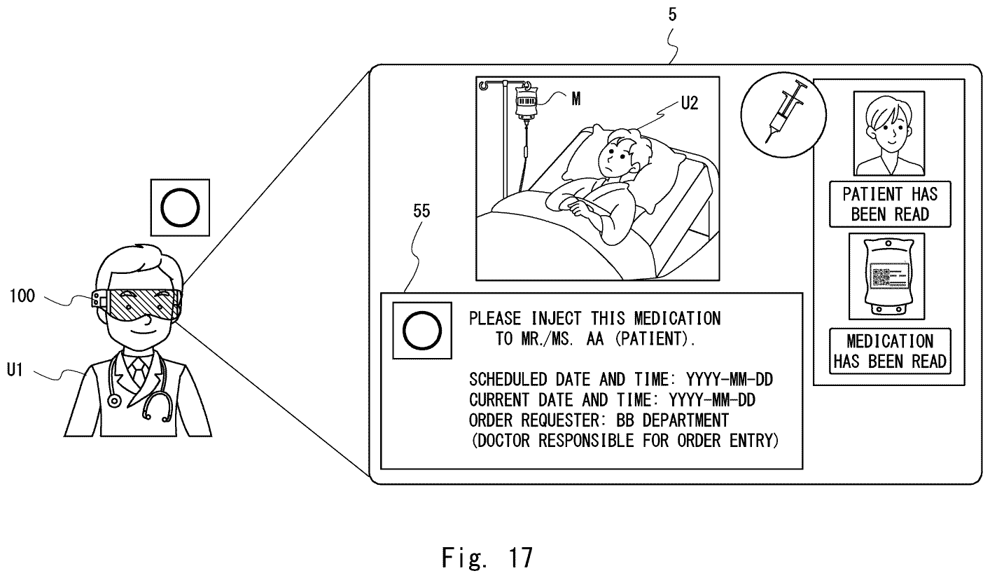

FIG. 17 is a diagram illustrating a display example of the wearer screen 5 of the AR glasses 100 in a case where the three-point authentication has succeeded. The wearer screen 5 includes a three-point authentication result 55. The three-point authentication result 55 is an example of display information indicating that the three-point authentication has succeeded. As a result, the wearer U1 of the AR glasses 100 can visually recognize that the wearer U1 can perform the medical act of administering the medication M to the patient U2 in front of the wearer U1 based on the three-point authentication result 55.

Therefore, the wearer U1 can perform the three-point authentication without using both hands, and inconvenience of the three-point authentication before the administration of the medication to the patient can be reduced. Therefore, even in an emergency or the like, the three-point authentication is hardly delayed, so that a risk of a medical accident can be reduced.

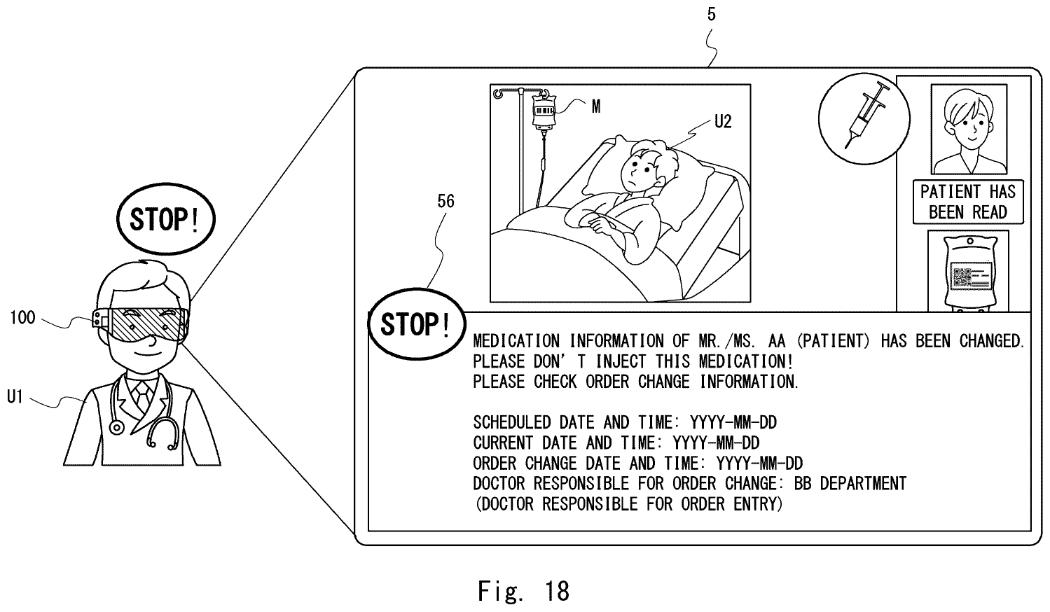

FIG. 18 is a diagram illustrating a display example of the wearer screen 5 of the AR glasses 100 in a case where the three-point authentication has failed. The wearer screen 5 includes a three-point authentication result 56. The three-point authentication result 56 is an example of display information indicating that the three-point authentication has failed. In an example of the three-point authentication result 56, the medication ID specified from the information code attached to the medication M is the past medication ID, and a message for inhibiting the administration of the medication M to the patient U2 is displayed. That is, a case in which the medication ID specified from the information code of the medication M imaged by the AR glasses 100 is different from the medication ID 421 of the medication registration information 420 searched in the medical information system 400, and coincides with the medication ID 4252 in the change history 425 is illustrated. As a result, the wearer U1 of the AR glasses 100 can visually recognize that the wearer U1 should not perform the medical act of administering the medication M to the patient U2 in front of the wearer U1 based on the three-point authentication result 56. The three-point authentication result 2122 is also recorded in the storage unit 210 of the authentication support server 200. Therefore, the administration of the medication M by maliciousness of the wearer U1 can be suppressed. In addition to the three-point authentication result 56, the wearer authentication result 2123 may indicate that the iris authentication of the wearer U1 has failed or the wearer U1 is not qualified for the medical act of administering the medication M. In this case, the wearer authentication result 2123 is also recorded in the storage unit 210 of the authentication support server 200 while being displayed on the wearer screen 5, the wearer U1 can avoid a moral issue such as impersonation by an unqualified person through misuse of a staff ID card or the like.

As described above, since it is originally necessary to perform an operation such as reading of an information code for the three-point authentication while holding a dedicated terminal at the time of administration of a medication to a patient, the hand is occupied, which is inconvenient. Therefore, there is a case where a confirmation result of the three-point authentication is recorded afterwards, and there is a problem that the risk of a medical accident is increased. In particular, in a case of an emergency or the like, there is also a problem that the three-point authentication is delayed, and as a result of which the risk of a medical accident is increased.

On the other hand, in the authentication support method according to the present disclosure, the wearer (the person scheduled to perform the medical act) wearing the AR glasses 100 can perform the three-point authentication in a state in which the hand is not occupied for the three-point authentication, and the three-point authentication result can be visually recognized. Therefore, the person scheduled to perform the medical act can perform the three-point authentication while verbally performing the medical interview with the patient and preparing for the medication administration. In particular, even in an emergency, it is possible to use both hands of the person scheduled to perform the medical act for the patient. Therefore, the inconvenience of the three-point authentication can be reduced, and a possibility that the three-point authentication is recorded afterwards can be reduced, so that a medical accident by the three-point authentication can be prevented. Therefore, the risk of a medical accident can be reduced. In addition, it is also a measure to prevent a moral problem such as impersonation of the medical practitioner by misuse of a staff ID card or the like.

### Third Embodiment

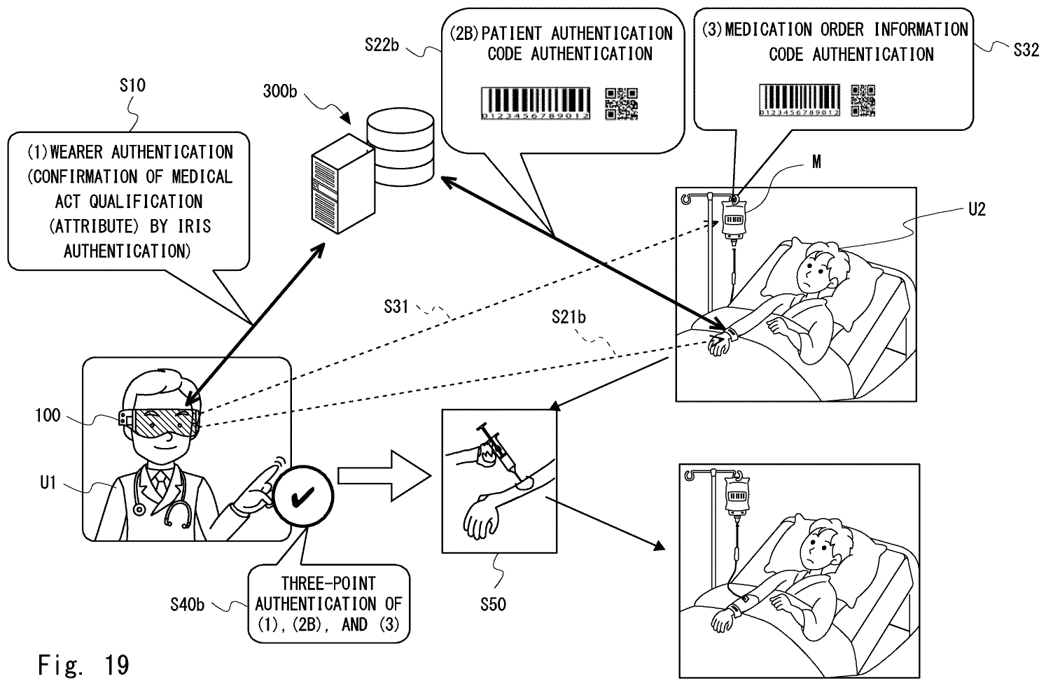

In a third embodiment, patient authentication in three-point authentication is code authentication. FIG. 19 is a diagram for describing a concept of the three-point authentication performed by a medical practitioner (wearer U1) wearing AR glasses 100. Hereinafter, differences from FIG. 4 described above will be mainly described. An authentication system 300b can make the face feature information 322 unnecessary in the patient DB 32 of the authentication system 300 of FIG. 3 described above. Instead, it is assumed that a patient U2 has a wristband on which an information code uniquely corresponding to a patient ID 321 of the patient U2 is posted and which is wound around the wrist of the patient U2.

First, similarly to FIG. 4, the AR glasses 100 perform “(1) wearer authentication” after the wearer U1 wears the AR glasses 100 (S10). In a case where the wearer authentication has succeeded, the wearer U1 directs the face (gaze) of the wearer U1 to the vicinity of the wrist of the patient U2. Therefore, an external camera (not illustrated) of the AR glasses 100 images the information code attached to the wrist of the patient U2 (S21b). Then, an authentication support server 200 performs identity confirmation (“(2B) patient authentication”) by the code authentication based on the imaged information code using the authentication system 300b (S22b). Similarly to FIG. 4, the AR glasses 100 perform “(3) code authentication” (S31 and S32). After steps S10, S22b, and S32, the AR glasses 100 display a result (here, “OK”) of the three-point authentication performed in (1), (2B), and (3) on a wearer screen (not illustrated) (S40b). As a result, the wearer U1 confirms that the three-point authentication has succeeded, and injects a medication M to the patient U2 (S50).

As described above, the same effects as those of the above-described second embodiment can be achieved by the third embodiment.

### Fourth Embodiment

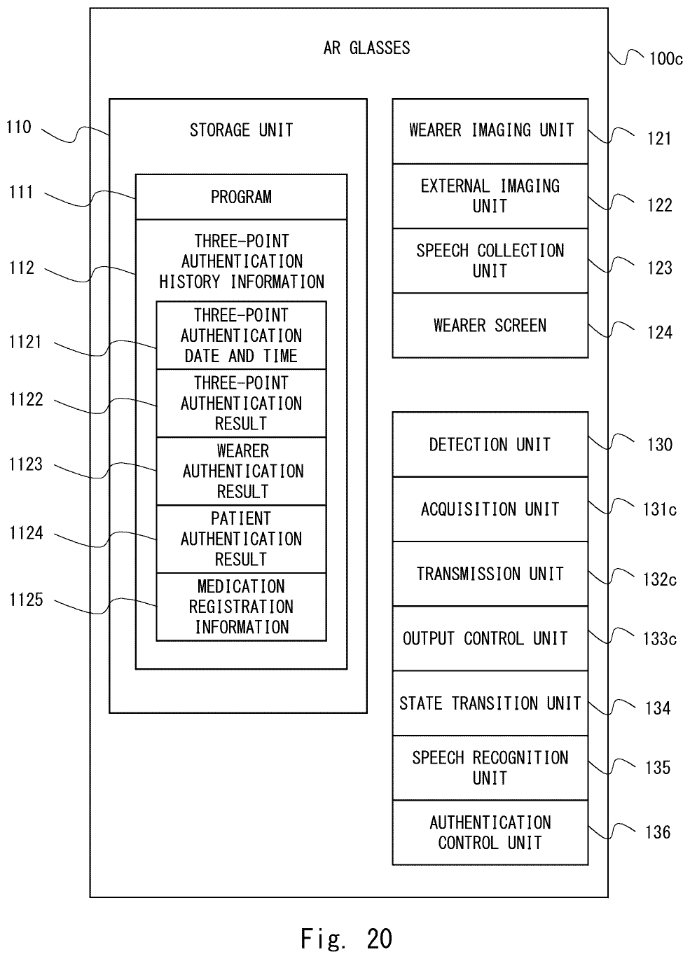

An authentication support apparatus according to a fourth embodiment incorporates the configuration and function of the authentication support server 200 described above in an eyewear-type wearable terminal. FIG. 20 is a block diagram illustrating a configuration of AR glasses 100c. As compared with FIG. 5 described above, in the AR glasses 100c, a storage unit 110, a state transition unit 134, a speech recognition unit 135, and an authentication control unit 136 are added, and an acquisition unit 131c, a transmission unit 132c, and an output control unit 133c are changed. The other configurations and an authentication system 300 and a medical information system 400 are similar to those of the above-described second embodiment. Therefore, a description of the configuration common to the AR glasses 100 is omitted below. Furthermore, a hardware configuration of the AR glasses 100c is similar to that of FIG. 6 described above.

The storage unit 110 corresponds to the memory 101 of FIG. 6 described above. A program 111 is obtained by changing the computer program stored in the memory 101 described above. The program 111 is a computer program in which processing of an authentication support method according to the present disclosure is implemented. Three-point authentication history information 112 is similar to the three-point authentication history information 212 of FIG. 7 described above. Therefore, a three-point authentication date and time 1121, a three-point authentication result 1122, a wearer authentication result 1123, a patient authentication result 1124, and a medication registration information 1125 included in the three-point authentication history information 112 correspond to the three-point authentication date and time 2121, the three-point authentication result 2122, the wearer authentication result 2123, the patient authentication result 2124, and the medication registration information 2125, respectively.

The acquisition unit 131c is an example of the first acquisition unit 11, the second acquisition unit 12, and the third acquisition unit 13 described above. In addition, the transmission unit 132c transmits an iris authentication request and a face authentication request to the authentication system 300 via a network N. Then, the acquisition unit 131c acquires the wearer authentication result and the face authentication result from the authentication system 300. Furthermore, the transmission unit 132c transmits a request for acquiring medical record information of a patient U2 to the medical information system 400 via the network N. Then, the acquisition unit 131c acquires the medical record information of the patient U2 from the medical information system 400 via the network N. In addition, the acquisition unit 131c acquires the medication registration information by specifying the medication registration information corresponding to medication information detected from an image related to a medication M captured by a wearer imaging unit 121. Specifically, the transmission unit 132c analyzes an information code to specify a medication ID, and transmits a medication registration information acquisition request including the specified medication ID to the medical information system 400 via the network N. Therefore, the acquisition unit 131c and the transmission unit 132c may implement some of the functions of the authentication control unit 222 described above.

The speech recognition unit 135 has a function similar to that of the speech recognition unit 223 described above.

In a case where a detection unit 130 detects a predetermined operation of a wearer U1 through the AR glasses 100c, the state transition unit 134 transitions an operation state of the AR glasses 100c to a three-point authentication execution state in which at least authentication information and the medication information can be acquired. Other functions of the state transition unit 134 are similar to those of the state transition unit 224 described above.

The authentication control unit 136 performs three-point authentication based on the acquired wearer authentication result, patient authentication result, and medication registration information.

After transitioning to the three-point authentication execution state by the state transition unit 134, the output control unit 133c displays a patient check field and a medication check field on a wearer screen 124. In addition, the output control unit 133c functions similarly to the output control unit 133 described above.

As described above, the same effects as those of the above-described second embodiment can be achieved by the fourth embodiment.

### OTHER EMBODIMENTS

The authentication support apparatus described above may detect a person region including the body of a person and a medication region including an information code of a medication from one image captured by the AR glasses 100 after transitioning to the three-point authentication execution state. In this case, the authentication support apparatus may further include a fourth acquisition unit that acquires biometric information of a patient as authentication information from the person region, and acquires medication information by analyzing the information code included in the medication region. Excessive authentication can be suppressed by acquiring the authentication information and the medication information from one image not before transitioning to the three-point authentication execution state but after transitioning to the three-point authentication execution state as described above.

The authentication support server 200 or the AR glasses 100c described above may incorporate some or all of the configurations and functions of the authentication system 300 and the medical information system 400.

While the present disclosure has been particularly shown and described with reference to embodiments thereof, the present disclosure is not limited to these embodiments. It will be understood by those of ordinary skill in the art that various changes in form and details may be made therein without departing from the sprit and scope of the present disclosure as defined by the claims. And each embodiment can be appropriately combined with at least one of embodiments.

Each of the drawings or figures is merely an example to illustrate one or more embodiments. Each figure may not be associated with only one particular embodiment, but may be associated with one or more other embodiments. As those of ordinary skill in the art will understand, various features or steps described with reference to any one of the figures can be combined with features or steps illustrated in one or more other figures, for example to produce embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

A (The) program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

Some or all of the above-described embodiments may be described as in the following Supplementary Notes, but are not limited to the following Supplementary Notes.

### (Supplementary Note A1)

An authentication support apparatus including:
a first acquisition unit configured to acquire a wearer authentication result indicating whether or not a wearer is a predetermined medical practitioner based on an iris authentication result of the wearer who is wearing the eyewear-type wearable terminal;
a second acquisition unit configured to acquire a patient authentication result based on authentication information of a patient imaged by the eyewear-type wearable terminal;
a third acquisition unit configured to acquire medication registration information indicating a correspondence between a medication specified based on medication information of the medication imaged by the eyewear-type wearable terminal and the patient who is an administration target; and
an output control unit configured to perform control to display, on a wearer screen of the eyewear-type wearable terminal, a three-point authentication result regarding permissibility of a medical act of administering the medication to the patient by the wearer based on the wearer authentication result, the patient authentication result, and the medication registration information.

### (Supplementary Note A2)

The authentication support apparatus according to Supplementary Note A1, further including a state transition unit configured to perform, in a case where a predetermined operation of the wearer through the eyewear-type wearable terminal is detected, transition of an operation state of the eyewear-type wearable terminal to a three-point authentication execution state in which at least the authentication information and the medication information are acquirable.

### (Supplementary Note A3)

The authentication support apparatus according to Supplementary Note A2, in which the state transition unit is configured to perform transition to the three-point authentication execution state in a case where the wearer authentication result indicates that the wearer is the predetermined medical practitioner and the predetermined operation of the wearer is detected.

### (Supplementary Note A4)

The authentication support apparatus according to Supplementary Note A2 or A3, in which the output control unit is configured to perform control to display, in a case where an image related to the patient or the medication is captured by the eyewear-type wearable terminal after transitioning to the three-point authentication execution state, a fact that the image is captured on the wearer screen.

### (Supplementary Note A5)

The authentication support apparatus according to any one of Supplementary Notes A1 to A4, in which the output control unit is configured to perform control to display, together with the three-point authentication result, display information describing a reason why the medical act is not permitted on the wearer screen of the eyewear-type wearable terminal in a case where the three-point authentication result indicates that the medical act is not permitted.

### (Supplementary Note A6)

The authentication support apparatus according to any one of Supplementary Notes A1 to A5, in which the output control unit is configured to operate, in a case where the three-point authentication result indicates the medical act is not permitted, the eyewear-type wearable terminal such that the wearer perceives a fact that the medical act is not permitted.

### (Supplementary Note A7)

The authentication support apparatus according to any one of Supplementary Notes A1 to A6, in which the eyewear-type wearable terminal incorporates the authentication support apparatus.

### (Supplementary Note A8)

The authentication support apparatus according to any one of Supplementary Notes A2 to A4, in which the state transition unit is configured to perform transition to the three-point authentication execution state in a case where utterance of a predetermined speech by the wearer is detected as the predetermined operation.

### (Supplementary Note A9)

The authentication support apparatus according to any one of Supplementary Notes A2 to A4, in which the state transition unit is configured to perform transition to the three-point authentication execution state in a case where a pressing operation by the wearer for instruction information for transition to the three-point authentication execution state displayed on the wearer screen is detected as the predetermined operation.

### (Supplementary Note A10)

The authentication support apparatus according to any one of Supplementary Notes A2 to A4, in which the state transition unit is configured to perform transition to the three-point authentication execution state in a case where utterance of a predetermined speech by the wearer after the wearer directs a gaze of the wearer through the eyewear-type wearable terminal for instruction information for transition to the three-point authentication execution state displayed on the wearer screen is detected as the predetermined operation.

### (Supplementary Note A11)

The authentication support apparatus according to any one of Supplementary Notes A2 to A4, in which the state transition unit is configured to perform transition to the three-point authentication execution state in a case where a change in gaze of the wearer toward an information code for three-point authentication posted at an administration place for the medication through the eyewear-type wearable terminal is detected as the predetermined operation.

### (Supplementary Note A12)

The authentication support apparatus according to any one of Supplementary Notes A2 to A4 or Supplementary Notes A8 to All, further including a fourth acquisition unit configured to acquire, in a case where a person region including a body of a person and a medication region including the information code of the medication are detected from an image captured by the eyewear-type wearable terminal after transitioning to the three-point authentication execution state, biometric information of the patient from the person region as the authentication information and acquire the medication information by analyzing the information code included in the medication region.

### (Supplementary Note A13)

The authentication support apparatus according to any one of Supplementary Notes A1 to A12, further including an authentication control unit configured to control iris authentication for an iris image of the wearer captured by a wearer camera of the eyewear-type wearable terminal, and determine whether or not the wearer is the predetermined medical practitioner based on the iris authentication result, in which the first acquisition unit is configured to acquire a determination result of the authentication control unit as the wearer authentication result.

### (Supplementary Note B1)

An authentication support system including:
an eyewear-type wearable terminal; and
an authentication support apparatus, in which
the eyewear-type wearable terminal is configured to transmit an iris image of a wearer who is wearing the eyewear-type wearable terminal, authentication information of an imaged patient, and medication information of an imaged medication to the authentication support apparatus,
the authentication support apparatus is configured to:
acquire a wearer authentication result indicating whether or not the wearer is a predetermined medical practitioner based on an iris authentication result for the iris image received from the eyewear-type wearable terminal;
acquire a patient authentication result based on the authentication information received from the eyewear-type wearable terminal;
acquire medication registration information indicating a correspondence between the medication specified based on the medication information received from the eyewear-type wearable terminal and the patient who is an administration target; and
transmit a three-point authentication result regarding permissibility of a medical act of administering the medication to the patient by the wearer to the eyewear-type wearable terminal based on the wearer authentication result, the patient authentication result, and the medication registration information, and
the eyewear-type wearable terminal is configured to display the three-point authentication result received from the authentication support apparatus on a wearer screen.

### (Supplementary Note C1)

An authentication support method executed by a computer, the authentication support method including:
acquiring a wearer authentication result indicating whether or not a wearer is a predetermined medical practitioner based on an iris authentication result of the wearer who is wearing an eyewear-type wearable terminal;
acquiring a patient authentication result based on authentication information of a patient imaged by the eyewear-type wearable terminal;
acquiring medication registration information indicating a correspondence between a medication specified based on medication information of the medication imaged by the eyewear-type wearable terminal and the patient who is an administration target; and
performing control to display, on a wearer screen of the eyewear-type wearable terminal, a three-point authentication result regarding permissibility of a medical act of administering the medication to the patient by the wearer based on the wearer authentication result, the patient authentication result, and the medication registration information.

### (Supplementary Note D1)

An authentication support program causing a computer to perform:
first acquisition processing of acquiring a wearer authentication result indicating whether or not a wearer is a predetermined medical practitioner based on an iris authentication result of the wearer who is wearing the eyewear-type wearable terminal;
second acquisition processing of acquiring a patient authentication result based on authentication information of a patient imaged by the eyewear-type wearable terminal;
third acquisition processing of acquiring medication registration information indicating a correspondence between a medication specified based on medication information of the medication imaged by the eyewear-type wearable terminal and the patient who is an administration target; and
output control processing of performing control to display, on a wearer screen of the eyewear-type wearable terminal, a three-point authentication result regarding permissibility of a medical act of administering the medication to the patient by the wearer based on the wearer authentication result, the patient authentication result, and the medication registration information.

Some or all of the elements (for example, the configurations and functions) described in Supplementary Notes A2 to A13 dependent on Supplementary Note A1 {e.g. apparatus} can also be dependent on Supplementary Note B1 {e.g. system}, Supplementary Note C1 {e.g. method}, and Supplementary Note D1 {e.g. program} by the same dependency relationship as Supplementary Notes A2 to A13. Some or all of the elements described in any supplementary notes may be applied to various hardware, software, recording means for recording software, systems, and methods.

What is claimed is:

1. An authentication support apparatus comprising:
at least one memory; and
at least one processor coupled to the at least one memory,
wherein the at least one processor is configured to:
acquire a wearer authentication result indicating whether or not a wearer is a predetermined medical practitioner based on an iris authentication result of the wearer who is wearing the eyewear-type wearable terminal;
acquire a patient authentication result based on authentication information of a patient imaged by the eyewear-type wearable terminal;
acquire medication registration information indicating a correspondence between a medication specified based on medication information of the medication imaged by the eyewear-type wearable terminal and the patient who is an administration target; and
perform control to display, on a wearer screen of the eyewear-type wearable terminal, a three-point authentication result regarding permissibility of a medical act of administering the medication to the patient by the wearer based on the wearer authentication result, the patient authentication result, and the medication registration information.

2. The authentication support apparatus according to claim 1, wherein the at least one processor is configured to perform, in a case where a predetermined operation of the wearer through the eyewear-type wearable terminal is detected, transition of an operation state of the eyewear-type wearable terminal to a three-point authentication execution state in which at least the authentication information and the medication information are acquirable.

3. The authentication support apparatus according to claim 2, wherein the at least one processor is configured to perform transition to the three-point authentication execution state in a case where the wearer authentication result indicates that the wearer is the predetermined medical practitioner and the predetermined operation of the wearer is detected.

4. The authentication support apparatus according to claim 2, wherein the at least one processor is configured to perform control to display, in a case where an image related to the patient or the medication is captured by the eyewear-type wearable terminal after transitioning to the three-point authentication execution state, a fact that the image is captured on the wearer screen.

5. The authentication support apparatus according to claim 1, wherein the at least one processor is configured to perform control to display, together with the three-point authentication result, display information describing a reason why the medical act is not permitted on the wearer screen of the eyewear-type wearable terminal in a case where the three-point authentication result indicates that the medical act is not permitted.

6. The authentication support apparatus according to claim 1, wherein the at least one processor is configured to operate, in a case where the three-point authentication result indicates the medical act is not permitted, the eyewear-type wearable terminal such that the wearer perceives a fact that the medical act is not permitted.

7. The authentication support apparatus according to claim 1, wherein the eyewear-type wearable terminal incorporates the authentication support apparatus.

8. An authentication support system comprising:
an eyewear-type wearable terminal; and
an authentication support apparatus, wherein
the eyewear-type wearable terminal includes:
at least one terminal memory; and
at least one terminal processor coupled to the at least one terminal memory,
the at least one terminal processor is configured to transmit an iris image of a wearer who is wearing the eyewear-type wearable terminal, authentication information of an imaged patient, and medication information of an imaged medication to the authentication support apparatus,
the authentication support apparatus includes:
at least one apparatus memory; and
at least one apparatus processor coupled to the apparatus memory, the at least one apparatus processor is configured to:
acquire a wearer authentication result indicating whether or not the wearer is a predetermined medical practitioner based on an iris authentication result for the iris image received from the eyewear-type wearable terminal;
acquire a patient authentication result based on the authentication information received from the eyewear-type wearable terminal;
acquire medication registration information indicating a correspondence between the medication specified based on the medication information received from the eyewear-type wearable terminal and the patient who is an administration target; and
transmit a three-point authentication result regarding permissibility of a medical act of administering the medication to the patient by the wearer to the eyewear-type wearable terminal based on the wearer authentication result, the patient authentication result, and the medication registration information, and
the at least one terminal processor of the eyewear-type wearable terminal is configured to display the three-point authentication result received from the authentication support apparatus on a wearer screen.

9. An authentication support method executed by a computer, the authentication support method comprising:
acquiring a wearer authentication result indicating whether or not a wearer is a predetermined medical practitioner based on an iris authentication result of the wearer who is wearing an eyewear-type wearable terminal;
acquiring a patient authentication result based on authentication information of a patient imaged by the eyewear-type wearable terminal;
acquiring medication registration information indicating a correspondence between a medication specified based on medication information of the medication imaged by the eyewear-type wearable terminal and the patient who is an administration target; and
performing control to display, on a wearer screen of the eyewear-type wearable terminal, a three-point authentication result regarding permissibility of a medical act of administering the medication to the patient by the wearer based on the wearer authentication result, the patient authentication result, and the medication registration information.
